(19) United States
(12) Patent Application Publication
(10) Pub. No.: US 2026/0007313 A1
(43) Pub. Date: Jan. 8, 2026

(54) Dual-Modal Photoacoustic and Fast Super-Resolution Ultrasound Imaging

(71) Applicant: The Board of Trustees of the University of Illinois, Urbana, IL (US)

(72) Inventors: Yun-Sheng CHEN, Champaign, IL (US); Yang ZHAO, Champaign, IL (US); Shensheng ZHAO, Urbana, IL (US)

(21) Appl. No.: 19/258,537

(22) Filed: Jul. 2, 2025

Related U.S. Application Data
(60) Provisional application No. 63/667,507, filed on Jul. 3, 2024.

(51) Int. Cl. A61B 5/00; A61B 5/1455; G06T 7/00

(52) U.S. Cl. CPC A61B 5/0095; A61B 5/0042; A61B 5/14551; G06T 7/0012; A61B 2562/043; G06T 2207/10132; G06T 2207/20084; G06T 2207/30104

(57) ABSTRACT

This disclosure describes examples of dual-modality imaging implementations involving both photoacoustic and fast super-resolution ultrasound localization imaging for non-invasive and non-superficial monitoring of both structural information and physiological activities/parameters in tissues. As an example, such dual-modality imaging may be particularly applied to the monitoring of structural information and physiological activities/parameters associated with the blood-brain barrier (BBB) in brain vascular structures that are subject to intervention/modulation (by, e.g., Focused Ultrasound, or FUS) for purposes of drug delivery from a blood flow to brain tissues.

### CROSS REFERENCE

This application is based on and claims the benefit of priority to U.S. Provisional Patent Application No. 63/667,507, filed on Jul. 3, 2024, which is herein incorporated by reference in its entirety.

### GOVERNMENT SUPPORT

This invention was made with government support under R01DK138939 awarded by the National Institutes of Health. The government has certain rights in the invention.

### BACKGROUND

### Technical Field

This disclosure generally relates relate to non-invasive and non-superficial imaging of tissues and is particularly directed to example dual-modality imaging implementations

### Background Technologies

Delivery of therapeutic agents to brain may be hindered by vascular Blood-Brian Barrier (BBB). The BBB may be modulated or intervened using external stimulus to facilitate such delivery. The modulation or intervention of the BBB needs to be closely monitored and adjusted in order to mitigate undesired side effects. Such monitoring may be based on various imaging techniques.

### BRIEF SUMMARY

This disclosure generally relates to non-invasive and non-superficial imaging of tissues and is particularly directed to example dual-modality imaging implementations involving both photoacoustic and high-resolution ultrasound localization imaging for non-invasive monitoring of both structural information and physiological activities/parameters in tissues. As an example, such dual-modality imaging may be particularly applied to a monitoring of structural information and physiological activities/parameters of blood-brain barrier (BBB) in brain vascular structures that is subject to intervention/modulation (by, e.g., focused ultrasound) for purposes of drug delivery from a blood flow to brain tissues.

In one example implementation, a method for imaging a target region surrounding a microvessel for blood is disclosed. The method may include injecting microbubbles in the microvessel having a low concentration in the target region; introducing a plurality of tracer agents in the microvessel in the target region; generating a sequence of ultrasound images based on ultrasound response from the microbubbles, the sequence of ultrasound images indicating a blood flow evolution in the microvessel; generating a sequence of optical pulses to induce acoustic responses from at least the plurality of tracer agents; and detecting the acoustic responses to form a sequence of photoacoustic images to monitor a diffusion evolution of the tracer agents through the microvessel, the sequence of photoacoustic images being time-interleaved with the sequence of ultrasound images at a frame rate of at least 10 frame per second.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example dual-modality imaging implementation for monitoring modulation/intervention of blood-brain barrier (BBB).

FIG. 2A and FIG. 2B illustrate an example deep learning model used to achieve a fast super-resolution ultrasound imaging.

FIG. 3A to FIG. 3J illustrate results for an example fast ultrasound imaging enabled by deep learning.

FIG. 4 illustrates multi-scale structural similarity of deep-learning-based ultrasound images with various duration of data acquisition time.

FIG. 5A to FIG. 5B illustrate example resolution measurement of ultrasound imaging.

FIG. 6 Illustrates microbubble counts as a function of injection during continuous perfusion using a syringe pump with the rate of 20 μL/min.

FIG. 7A to FIG. 7C illustrate a comparison of blood speed distribution using 100 sec acquisition and 4 second acquisition from FIG. 8B below. The Chi-Squared distance is calculated to measure the dissimilarity between two histograms.

FIG. 8A to FIG. 8J illustrate example 3D functional ultrasound imaging with mechanical scanning.

FIG. 9A and FIG. 9B illustrate analysis of the velocity profile showing a Poiseuille-like flow profile.

FIG. 10 illustrates example analysis of brain blood speed in a whole brain and anatomical brain regions of Isocortex and Midbrain.

FIG. 11A to FIG. 11J illustrate example dual-modal photoacoustic ultrasound localization (PAUL) imaging that reveals structural and functional information of a mouse brain in an oxygen-challenge test.

FIG. 12A to FIG. 12D illustrate Hb and HbO₂ change of a mouse brain in an oxygen-challenge test.

FIG. 13 shows percentage change of brain blood speed (black) and microbubble (MB) flux (red) over two periods of hyperoxia-and-hypoxia cycles.

FIG. 14A to FIG. 14J illustrate dual-modal 3D PAUL detected Blood-Brain Barrier Disruption.

FIG. 15A to FIG. 15C illustrate focus ultrasound characterization.

FIG. 16A to FIG. 16D illustrate in vivo indocyanine green (ICG) distribution in a mouse brain before and after 30 minutes, 110 min and 170 min of BBB disruption with focused ultrasound. The brain microvascular image merged with ICG signals is collected after 110 min of BBB disruption.

FIG. 17A to FIG. 17B illustrate coronal views of 3D PA images of ICG distribution in the brain a, before and b, 110 minutes after BBB disruption. The images cover the region from bregma −1.9 mm to −4.15 mm. The atlas map at bregma −3.3 mm (brown line) guides the identification of brain regions. The scale bar is 1 mm. The scanning step size is 50 μm.

FIG. 17C to FIG. 17D illustrate coronal views of PA image of ICG with the region at bregma −3.3 mm (a), before and b, 110 minutes after BBB disruption. The scale bar is 1 mm. The images cover the region from bregma −1.9 mm to −4.15 mm. The atlas map at bregma −3.3 mm (white line) guides the identification of brain regions

FIG. 18A to FIG. 18J illustrate dual-modal 3D PAUL for detecting the relation between blood flow speed and FUS-BBB opening.

FIG. 19 illustrates velocity distribution in the left and right hemispheres of a mouse with FUS-mediated BBB disruption at the brain region from bregma −1.9 to −4.15 mm.

FIG. 20A to FIG. 20D illustrate example in vivo photoacoustic imaging for revealing blood oxygenation.

FIG. 21A to FIG. 21D illustrate validation of ICG as BBB permeability tracers.

FIG. 22A to FIG. 22C illustrate false negative rate (FNR) and false positive rate (FPR) in DL-UL imaging. A-B, the difference images of Sparse UL and ground truth (GT), DL-UL and GT, respectively, where green colour indicates false negative, red indicates false positive and yellow indicates true positive. C, The comparison of DL-UL and Sparse-UL in terms of accuracy, FNR and FPR.

FIG. 23A and FIG. 23B illustrate false negative rate (FNR) and false positive rate (FPR) in DL-UL imaging under oxygen challenge. A, The performance of Sparse-UL and DL-UL with 100% oxygen inhalation. B, The performance of Sparse-UL and DL-UL with 3% oxygen inhalation.

FIG. 24A to FIG. 24F illustrate visualization of single microbubbles. A, B, the microbubble images after SVD declutter filtering. The localized microbubble positions are indicated by red stars. C,D, Digitalized microbubble distribution image in one ultrasound frame generated by localized microbubble positions. E,F, Zoom-in microbubble distribution.

FIG. 25 illustrates coronal view of a 3D microbubble flux map of a mouse brain. The scanning step size is 50 μm. The scale bar is 2 mm.

FIG. 26A to FIG. 26E illustrate quantitative PA phantom tube imaging. A, Ultrasound tube image, where the cross sections of two tube profiles are indicated by white dash circles. The depth of the tube is 3.5 mm and 16.2 mm, respectively. B-C, PA tube image at 720 nm and 780 nm wavelength. The ICG solution with 0.5 mg/mL was filled in the tubes. D-E, PA signal as a function of ICG concentration inside tube 1 and 2.

FIG. 27 illustrates tissue oxygenation and blood flow change under oxygen challenge.

FIG. 28 illustrates the Evans blue dye distribution in the brains of mice 2 hours after being treated with different FUS doses. Both low and high FUS dose cases show the accumulation of Evans blue dye at the region of the FUS exposure, demonstrating the successful BBB opening. However, the second case shows visible blood bleeding caused by blood vessel damage.

FIG. 29A and FIG. 29B illustrates histology evaluation of FUS medicated BBB opening. A, Histology images of mouse brains in different groups 2 hour after FUS-induced BBB opening. B, Zoom-in histology images highlighted by black boxes indicated in a.

FIG. 30A to FIG. 30F illustrate cell apoptosis evaluation after FUS-mediated BBB disruption. A-B, Fluorescence images of DAPI (stain nuclear), Alexa Fluor® 488 (stain TUNEL assay) and its fusion in brain samples from Group 1 and Control group (no FUS activation). C-D, Zoom-in DAPI and AF 488 signal in the left and right regions in Group 1 and Control group, indicated by white boxes in A-B. E, Quantitative comparison of DAPI signal in left and regions from Control groups and Group 1. N=4. ns: not significant difference. F, Quantitative comparison of AF 488 signal in left and regions from Control groups and Group 1. N=4. ns: not significant difference.

FIG. 31A to FIG. 31D illustrate the effectiveness of FUS medicated BBB disruption under different conditions. A, The photograph of harvest brain in Group 1. B, Fluorescence image of ICG distribution in whole brain and one coronal slice after FUS medicated BBB disruption in Group 1. C, The photograph of harvest brain in Group 2. The white arrow indicates blood bleeding. D, Fluorescence image of ICG distribution in the whole brain and one coronal slice after FUS medicated BBB disruption in Group 2.

FIG. 32A to FIG. 32D illustrate evaluation of BBB disruption in UL imaging. A, Brightfield and Evans blue fluorescence image of brain sample after whole brain UL imaging (Imaging group). B, Brightfield and Evans blue fluorescence image of brain sample in Control group. C. Quantitative comparison of Evans blue fluorescence intensity in both groups. D, Whole brain IVIS images of Evans blue distribution in both groups.

### DETAILED DESCRIPTION

Various example implementations will now be described in detail hereinafter with reference to the accompanied drawings, which form a part of the present disclosure. The systems, devices, and methods disclosed herein may, however, be embodied in a variety of different forms and, therefore, the disclosure herein is intended to be construed as not being limited to the embodiments set forth below. Further, the disclosure may be embodied as methods, components, and/or platforms in addition to the disclosed devices and systems. Accordingly, embodiments of the disclosure may, for example, take the form of hardware, software, firmware or any combination thereof.

In general, terminology may be understood at least in part from usage in its context. For example, terms, such as “and”, “or”, or “and/or,” as used herein may include a variety of meanings that may depend at least in part upon the context in which such terms are used. Typically, the term “or”, if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for the existence of additional factors not necessarily expressly described, again, depending at least in part on context.

Many other modifications of the implementations above may be made to adapt a particular situation or material to the teachings without departing from the scope of the current disclosure. Therefore, it is intended that the present methods and systems not be limited to the particular embodiments disclosed, but that the disclosed methods and systems include all embodiments falling within the scope of the appended claims.

The example embodiments below relate to dual-modality imaging implementations involving both photoacoustic and fast super-resolution ultrasound localization imaging for non-invasive and non-superficial monitoring of both structural information and physiological activities/parameters in tissues. As an example, such dual-modality imaging may be particularly applied to monitoring of structural information and physiological activities/parameters associated with blood-brain barrier (BBB) in brain vascular structures that is subject to intervention/modulation (by, e.g., Focused Ultrasound, or FUS) for purposes of drug delivery from a blood flow to brain tissues.

In further detail, delivery of therapeutic agents to the brain from blood flow is hindered/complicated by the BBB at brain vascular walls. The FUS technique offers a non-invasive method or approach to modulate BBB permeability, thereby generating or enabling transient openings in the BBB for therapeutic agent delivery from a blood stream to surrounding brain tissues. Techniques for BBB modulation/intervention including but not limited to FUS play a critical role in therapeutic agent delivery for treating brain-related health conditions such as Parkinson's disease, Alzheimer's disease, epilepsy, and brain cancer.

However, excessive BBB disruption may risk cerebral damages and neurological symptoms. As such, this process, including both the opening of the BBB and the diffusion/delivery of the therapeutic agents must be precisely controlled and/or monitored to prevent severe BBB disruption and the resultant unintended neurological side effects. Current imaging techniques often lack the ability to simultaneously assess detailed hemodynamic changes and BBB integrity across the whole brain. For example, the current imaging techniques often lack the resolution necessary to accurately capture the brain's response to BBB disruption or cannot effectively visualize the therapeutic agents' diffusion process across the BBB. These current techniques lack the ability to provide detailed hemodynamic data in tandem with BBB integrity assessments, or are incompatible with FUS interventions. Thus, whether the BBB disruption is excessive and/or details of the risks of the BBB opening is not easily ascertained using these existing imaging techniques.

In the disclosure below, example implementations involving a FUS-compatible and multi-parametric photoacoustic/ultrasound localization (PAUL) imaging method employing a clinical ultrasound linear array are described. These dual-modality techniques combine the strength of both photoacoustic imaging and super-resolution ultrasound imaging to simultaneously monitor both the detailed cerebral hemodynamic changes and critical physiological response to BBB permeability occurring in the brain following BBB modulation/intervention and the diffusion of therapeutic agents through an intact skull by capturing changes in blood flow, volume, and oxygenation, as well as mapping the distribution of BBB permeability tracers in addition to vascular structural information. Such dual-modality techniques enable a comprehensive whole brain imaging through the intact skull with mechanical scanning with visualization of brain microvascular structures at a resolution of, for example, ˜22 μm. These techniques accelerate whole-brain data acquisition to much shorter time, e.g., in just 16 minutes—25 times faster than conventional UL imaging with a linear array. Further, by leveraging Deep Learning (DL)-accelerated PAUL imaging, it is revealed that BBB modulation is associated with reduced blood flow, especially in regions exhibiting high tracer retention. These findings demonstrate the potential of the dual-modality imaging technique for non-invasive, whole-brain monitoring of BBB disruption caused by FUS, offering a powerful tool for advancing the understanding and clinical management of BBB dynamics. The implementations disclosed herein inform functioning, risks, management, evaluation, and the like to the BBB intervention/modulation procedures.

While the dual-modality imaging implementations below are described in the context of BBB monitoring, such implementations are not so limited and can be applied to any other contexts and applications where both structural and dynamical functional/physiological information are important.

### Brain Blood Barrier (BBB) and Focused Ultrasound (FUS) Modulation/Intervention

The orchestration of cerebral blood flow (CBF), oxygenation, and the integrity of the blood-brain barrier (BBB) in a central nervous system (CNS) are foundational to both its normal functioning and its vulnerability in pathological states. The Brain Blood Barrier (BBB) resides at brain vascular walls and acts as a critical gatekeeper, maintaining the CNS's internal environment by selectively restricting the passage of substances. The gatekeeper nature of BBB, however, poses challenges in delivering therapeutic agents to the brain from the blood steam, preventing 95% of therapeutic molecules from progressing toward target areas.

Techniques to transiently modulate/intervene with the BBB permeability, especially the non-invasive approaches such as focused ultrasound (FUS), have emerged as a promising strategy for enhancing drug delivery to the brain. During such modulation/intervention, the BBB may temporally open, allowing for passage of the therapeutic agents to the brain tissues.

However, permanent BBB disruption is unwanted. Particularly, permanent BBB disruption has been attributed as a common denominator in a wide array of neurological disorders, including stroke, Alzheimer's disease, Parkinson's disease, Huntington's disease, and epilepsy, highlighting its pivotal role in the health and pathology of the brain. Given that BBB disruption is closely linked to these neurological disorders, modulating it by, e.g., FUS, without careful control may pose potential risks, including unintended neurological side effects. This delicate balance underscores the need for monitoring and precise control of BBB modulation/intervention to avoid/mitigate potential risks such as irreversible opening and other permanent interruptions of the BBB. Such monitoring would provide guidance to, for example, the FUS procedure in order to mediate these risks.

Such monitoring, in one aspect, may include high-resolution microscopy for rendering structural information. Some traditional structuring imaging methods for evaluating the BBB permeability may rely on in vitro cell membrane assays and ex vivo tissue analysis. These methods, including high-resolution microscopy techniques such as immunohistochemistry, nonlinear optical microscopy, and electron microscopy, provide detailed insights into BBB structure down to the sub-cellular level. However, they fall short of allowing for repeated measurements in the same subject, which is essential for tracking dynamic changes in BBB permeability and other physiological parameters caused by the changes due to BBB modulation/intervention described above.

Such monitoring, in another aspect, may further need to involve monitoring of functional/physiological activities changes/responses that may not be sufficiently ascertained from structural imaging. For example, it has been shown that the disruption of BBB is associated with dysregulation of CBF and reductions of blood oxygenation, which may need to be tracked beyond structure imaging.

Various clinical imaging technologies have been developed to study BBB and cerebral vasculature. For example, magnetic resonance imaging (MRI), with modalities such as phase-contrast, contrast-enhanced MRI, and blood-oxygen-level-dependent (BOLD) MRI, has been used in preclinical and clinical BBB studies. While these MRI techniques can non-invasively image cerebral vasculature, measure blood oxygenation, and estimate BBB permeability, and has been used to show that FUS-mediated BBB modulation induces a heterogeneous cerebral vascular response, its resolution, especially for functional MRI, is still insufficient for detailed analysis. For another example, positron emission tomography, while offering a wide selection of tracers for BBB permeability studies, is still hampered by its poor spatial resolution and a need for radioactive labeling.

For another example, techniques such as intravital optical microscopy and functional photoacoustic (PA) microscopy, while allowing for real-time monitoring of BBB modulation and offer high-resolution insights, are nevertheless restricted to superficial brain areas. For another example, PA tomography, while being effective in tracking cerebral oxygenation and dye tracer dispersion in BBB permeability modulation studies in deep brain, is incapable of precise imaging of CBF. For another example, super-resolution PA imaging has emerged to improve the spatial resolution of blood flow imaging. However, these techniques rely on specialized imaging agents and are constrained by slow imaging speed due to low laser repetition rates, limiting their ability to capture rapid vascular dynamics. For yet another example, Ultrasound localization (UL) imaging partially addresses these issues by detailing microvascular changes, yet it primarily visualizes vascular responses without directly imaging BBB permeability tracers and cerebral oxygenation.

To address this gap, it is proposed to integrate PA and UL imaging, a dual-modal technique that combines the strengths of PA and UL imaging. PAUL has demonstrated its ability to provide comprehensive structural, physiological, and molecular insights through kidney imaging and functional neuroimaging, highlighting its potential for BBB studies. However, applying PAUL to monitor whole-brain BBB disruption remains technically challenging, particularly when using linear array transducers. Linear arrays require sequential scanning and prolonged data acquisition (DAQ) to capture a single dual-modal frame, rendering whole-brain imaging impractically slow. Although increasing microbubble concentrations can shorten the acquisition time, sustained high-dose injections during scanning introduce safety concerns due to established microbubble dosage limits. This presents a critical trade-off between reducing scan time and ensuring safe microbubble administration, underscoring the need for optimized imaging strategies that balance speed, resolution, and safety in whole-brain BBB modulation studies.

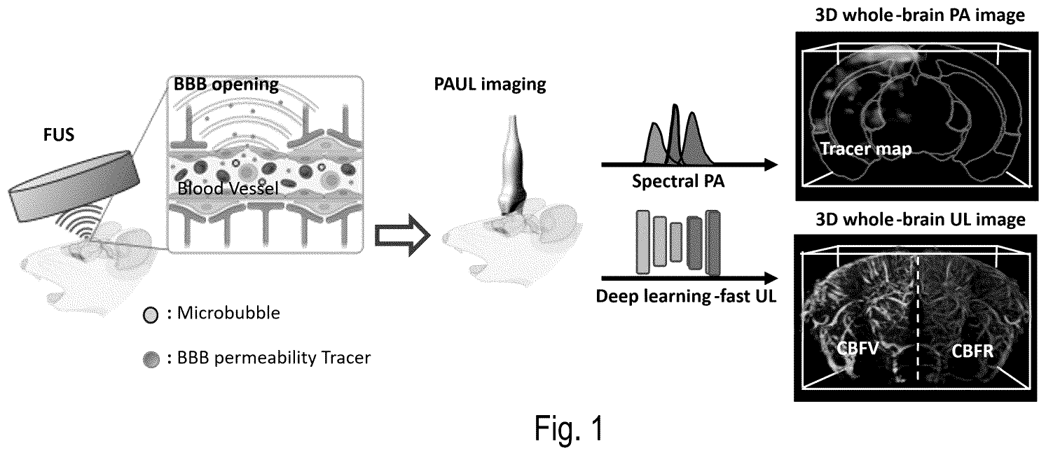

To overcome these challenges, deep learning (DL)-enhanced multi-parametric PAUL whole-brain imaging, a comprehensive tool for BBB modulation monitoring, is developed. The DL model, based on a generative network, effectively reconstructs sparse imaging data into a complete vascular map without the need for high microbubble concentration. This advancement not only ensures prolonged imaging times but also enhances the safety and feasibility of whole-brain 3D imaging with a linear array transducer. It substantially improves compatibility with FUS-mediated BBB opening and enables PAUL imaging to achieve whole-brain high-resolution visualization of cerebral microvasculature structure, hemodynamic changes, blood oxygenation, and tracer movement across the BBB (FIG. 1).

The disclosure below reveal that the DL-enhanced PAUL imaging achieves the first whole-brain imaging with a linear array, surpassing the resolution of traditional ultrasound methods in depicting cerebral vasculature and capturing hemodynamic changes with precision. These changes, particularly in response to varying oxygen levels, enable a direct link to be established between oxygen delivery and vascular reactivity-key indicators essential for monitoring BBB modulation. Moreover, PAUL imaging proves to be an invaluable asset in guiding and evaluating non-invasive BBB modulation techniques, such as those facilitated by FUS. This capability allows for the detailed tracking of tracer diffusion and cerebrovascular responses, illuminating the complex reactions of the brain to such interventions.

### Dual-Modality Multi-Parametric Photoacoustic/Ultrasound Localization (PAUL) Imaging-General Implementations

As described above, in order to dynamically detect the structural, substance permeabilities (e.g., therapeutic agent permeabilities), and physiological parameters such as oxygenation and the like in BBB modulation/intervention, a new imaging technical or modality may need to be developed. Such imaging modality may need to achieve high resolution and be non-intrusive, yet it is not limited to superficial imaging.

In some example implementations, to achieve such an imaging goal and to bridge the gap in various other imaging techniques described above, a multi-parametric photoacoustic-ultrasound localization (PAUL) imaging configuration is described below. As one example application, such imaging can be performed through an intact skull to monitor the blood-brain barrier (BBB) and observe the structural, functional, and physiological changes in the brain during BBB modulation and intervention. Such imaging implementations may further incorporate deep learning (DL) techniques to enhance imaging speed, particularly with respect to the ultrasound (UL) imaging aspect. The example imaging implementations combine photoacoustic (PA) imaging with deep learning-enhanced fast UL imaging into a dual-modality system that leverages the strengths of both techniques. By doing so, the example PAUL imaging implementations help achieve fast high-resolution visualization of the cerebral microvasculature's structure, hemodynamic changes, blood oxygenation, and the movement of tracers across the BBB.

Such PAUL imaging implementations may surpass the resolution of traditional ultrasound methods in, for example, depicting cerebral vasculature and capturing hemodynamic changes with precision. These changes, particularly in response to varying oxygen levels, enable a direct link to be established between oxygen delivery and vascular reactivity as key indicators essential for monitoring BBB modulation. Such PAUL imaging implementations may be used for evaluating non-invasive BBB modulation techniques, such as those facilitated by FUS, allowing for detailed tracking of tracer diffusion and cerebrovascular responses and monitoring of complex reactions of the brain to such interventions.

In some example implementations, the super-resolution UL imaging above of the dual-modality PAUL imaging may be performed to provide vascular and other structural information during, e.g., the BBB intervention. In particular, ultrasound may be configured to impinge through the imaging area (portion of the brain, for example) and detected by ultrasound detectors in order to extract spatial imaging information. In addition, microbubbles may be introduced/injected into the blood flow or otherwise generated in the blood flow. Such microbubbles may also be imaged via ultrasound. Because the microbubbles flow with the bloodstream, their image can carry information related to the blood flow, which may be dynamically changed during the BBB modulation/intervention. Such UL imaging information thus can provide BBB functional information and vascular response information that may not be capturable by the slow-changing vascular structural information. For example, during the BBB intervention using FUS, vascular responses may include changes that affect the blood flow. Such responses may be detected and captured by imaging the microbubbles.

In some implementations, the density of the microbubbles may be controlled to a low level, e.g., below a certain threshold concentration level. Imaging with higher concentration of microbubbles may help obtaining faster UL imaging. However, excessive bubbles may affect the FUS process for the BBB modulation/intervention, and the UL imaging of high-concentration bubbles may be saturated such that imaging information extraction is inaccurate. UL imaging with low-concentration microbubbles thus provides the benefit of minimal impact on the BBB modulation/intervention and more accurate imaging. However, UL imaging with low concentration microbubbles may be slow in speed because multiple UL images may need to be taken and superposed in order to generate high-resolution full images.

In some example implementations, a deep learning model (including multiple layers of neural networks, for example) may be constructed and trained to process an UL image taken with low-concentration microbubbles (e.g., 10⁸ per ml, or less than 10⁷ per ml) to generate a full super-resolution image. In such a manner, the super-resolution UL may be taken at a much faster time scale and be synchronized with the photoacoustic imaging described above in an interleaving manner in time in order to register the measured imaging information between the two modalities, as described in further detail below. Further details are provided below.

In some example implementations, the photoacoustic (PA) imaging aspect of the PAUL imaging above may be performed by introducing optical agent tracers in the blood flow and by imaging such tracers by detecting sound generation as a result of optical absorption by the tracers. For example. A short optical pulse, e.g., a short laser pulse, may be tuned to an absorption line or band of the tracers and spatially flood the imaging area. The tracers may subsequently absorb the optical pulses and heat up, leading to thermal modulation that generates a sound signal following the optical pulse, which can be detected by an array of sound detectors. Such sound and sound detectors may be in the ultrasound frequency range. The PA spatial imaging is thus achieved via the sound detector array (or acoustic detector array). The sound/acoustic detector array may be gated according to the timing of the excitation optical pulse. Because the tracer agent can flow through the BBB openings during the BBB modulation/intervention process, such PA image thus may provide information about the BBB opening.

For the PA imaging aspect above, the optical pulse may be additionally tuned to one or more other wavelengths or spectral bands associated with elements in the blood flow that correlate with the blood oxygenation level. These elements may absorb the optical pulse and convert the energy into sound for detection by the sound/acoustic detector array above. As such, a spatially resolved oxygenation profile may be obtained via the PA imaging modality. Further details of these implementations are provided below.

The UL imaging and the PA imaging of the dual model PAUL imaging above may be sequenced or interleaved to monitor the BBB modulation/intervention process. In some example implementations, the following example sequence may be employed:
Start the FUS process for BBB modulation/intervention.
Repeating the following sequence in time:
   Inject PA tracer agent in the blood stream.
   Inject low-concentration microbubbles in the blood stream.
   Send UL pulse through the detection area.
   Recording UL image.
   Tune an optical source to generate an optical pulse at a first wavelength associated with the absorption line of the PA tracer agent and flood the detection area with the optical pulse.
   Record PA image of the tracer agent indicating BBB opening information using the acoustic/sound detector array.
   Tune the optical source to generate at least another optical pulse at another wavelength associated with absorption line of an element associated with the oxygenation level and sending the another optical pulse through the detection area.
   Record another PA image using the acoustic/sound detector array for monitoring the oxygenation profile.
   Perform image processing using the trained deep learning model to generate super-resolution UL image (which provide vascular structural information and blood flow information) and spectrum unmixing to extract oxygenation profile and tracer distribution.

The PA and UL time-interleaving sequences above are merely examples. The steps in each repetition may be ordered in other manners. Some of the steps may be performed at the same time as needed. For example, the PA imaging may alternatively be performed before the UL imaging. In some example implementations, the UL imaging may be performed between the two PA processes (one for the PA tracer agent, and one for the oxygenation profile). In some other example implementations, the two PA imaging processes may be performed in a single imaging step by sending the two laser pulses simultaneously. In some example implementations, the microbubbles and/or the tracer agent may be continuously and steadily supplied to the detection area via the bloodstream.

The above sequences provide a dynamic measurement of the various structural and parametric information during the BBB modulation/intervention.

Each of the PA and/or UL images above may be a collection of slices of 2D images forming a 3D image.

FIG. 1 illustrates the general implementations of the PAUL imaging technique above, particularly for monitoring BBB modulated/intervened by focused ultrasound (FUS), where the FUS is applied and the dynamics of the structural, functional, permeability, and other parametric information is imaged via the PAUL imaging including both fast super-resolution UL imaging and PA imaging.

### Dual-Modality Multi-Parametric Photoacoustic/Ultrasound Localization (PAUL) Imaging-More Details and Results

UL imaging above represents an evolution of traditional ultrasound imaging. It offers enhanced spatial resolution, achieving ten-fold improvements (10-50 μm) within several centimeters of depth. UL imaging accurately maps microvascular flow by tracing contrast agents, such as the microbubbles above. Complementing this, PA imaging above detects signals from optically absorbing molecules, enabling tracking of diffused tracers through a disrupted BBB and measuring cerebral blood oxygen levels. The integration of PA and UL imaging is expected to seamlessly register dual-contrast images, effectively monitoring tracer diffusion, tissue oxygenation, microvasculature, and vascular flow changes related to BBB permeability, offering a comprehensive tool for BBB studies.

Integrating UL with PA imaging presents technical challenges, primarily due to the slow nature of UL imaging, which takes over a minute to construct a single 2D image frame. This slow acquisition rate further hinders the collection of consecutive 2D images necessary for 3D stacking, especially when using a clinical linear array ultrasound imaging system.

### UL Imaging and Results

To address these issues, UL imaging employs advanced localization algorithms such as compressed sensing or machine learning to enhance the localization efficiency of microbubbles, thus accelerating the acquisition speed of UL imaging. These approaches require an increase in the microbubble dose. However, it is still unclear how this high dosage will affect the hemodynamics of the brain and BBB integrity. Additionally, even with highly concentrated microbubbles, the narrow cerebral blood microvessel has a slow blood flow speed, which saturates and limits the maximum number of microbubbles in the imaging view. The localization number of microbubbles is thus not necessarily linearly increased with the increment of injected microbubble concentrations. These factors must be carefully considered when developing fast UL imaging to monitor FUS BBB modulation.

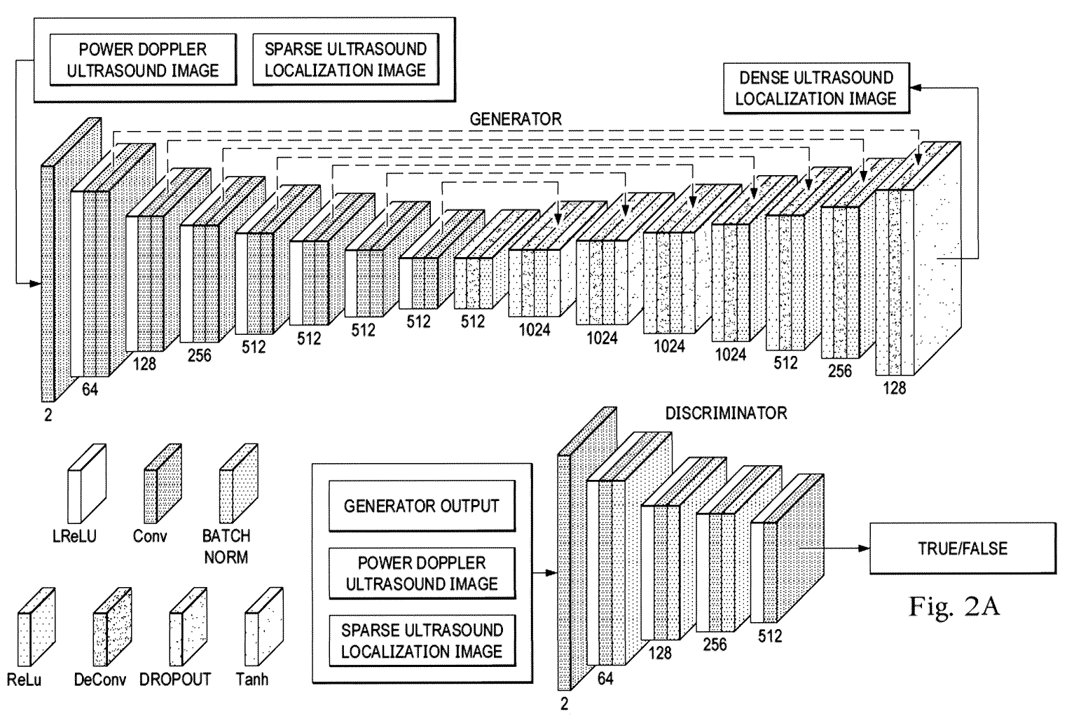

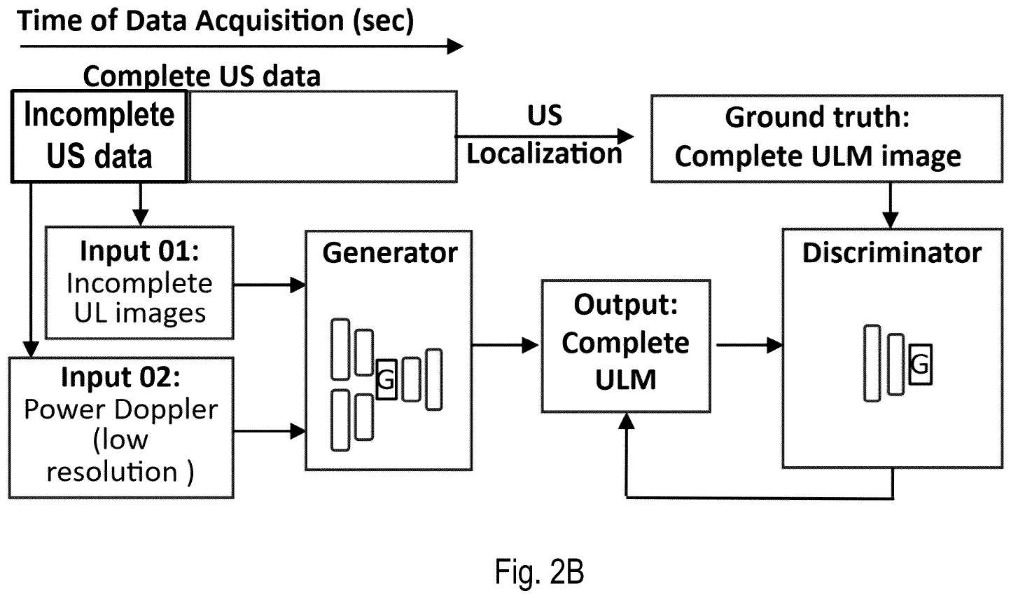

As described above, a deep-learning (DL) approach to construct a complete super-resolution vascular map from incomplete UL data during a brief recording, eliminating the need for elevated microbubble concentrations in the brain. Different from traditional techniques that rely on differentiating densely overlapping microbubbles, the implementations disclosed herein modified an example pix2pix model, an image-to-image translation framework based on conditional Generative Adversarial Networks (cGANs), a deep learning architecture for generating data without complex probability density functions. As illustrated in FIG. 2A and FIG. 2B, a customized example cGAN features a U-Net-based generator and a five-layer discriminator. The inputs of DL model are sparse UL images and power Doppler images reconstructed from the same datasets with short DAQ. The model processes the inputs using an encoder with down-sampling convolution layers, batch normalization, and leaky rectified linear units (ReLUs). The decoder then upscales these inputs through convolution layers and nearest-neighbor interpolation, culminating in a Tanh-activated final layer that produces dense UL images. The quality of these images may be further enhanced by batch normalization and a 0.5 dropout rate. Once trained, the DL model can reconstruct a complete UL image within 0.1 seconds, significantly faster than conventional UL methods and other computational acceleration techniques, such as compressed sensing.

For training of the example deep learning model above, ground truth images are first abstained. Conventional in vivo UL images are captured from mouse brains through intact skulls under low-concentration microbubble injection (e.g., 1×10⁸ bubbles/mL). Specifically, plane-wave ultrasound imaging is performed at 500 Hz with 7 angles ranging from −6° to 6°. Multi-angle compounding beamforming followed by singular value decomposition (SVD) filters is then utilized to differentiate moving microbubble signals from static tissue clutter, a process known as decluttering. The precise positions of microbubbles in each ultrasound frame may be determined using the point spread function (PSF) of the imaging system, identified via Gaussian fitting of isolated microbubble signals. This information may be cross-correlated across frames for accurate microbubble localization. UL images exhibiting signal saturation above a certain threshold are selected as ground truth. Producing such images may require a number of, e.g., at least 100 seconds of data acquisition (DAQ) for a 2D frame, equivalent to 50,000 B-mode ultrasound images.

To create the input data for network training, a subset of sequential UL images with 4-second DAQ is randomly selected to generate incomplete sparse UL images. Parallelly, power Doppler ultrasound images are generated from the same dataset. These images are then divided into batches and shuffled for training. The network may be trained using these sparse UL images and power Doppler images, employing loss validation methods to prevent overfitting and ensure network generalization.

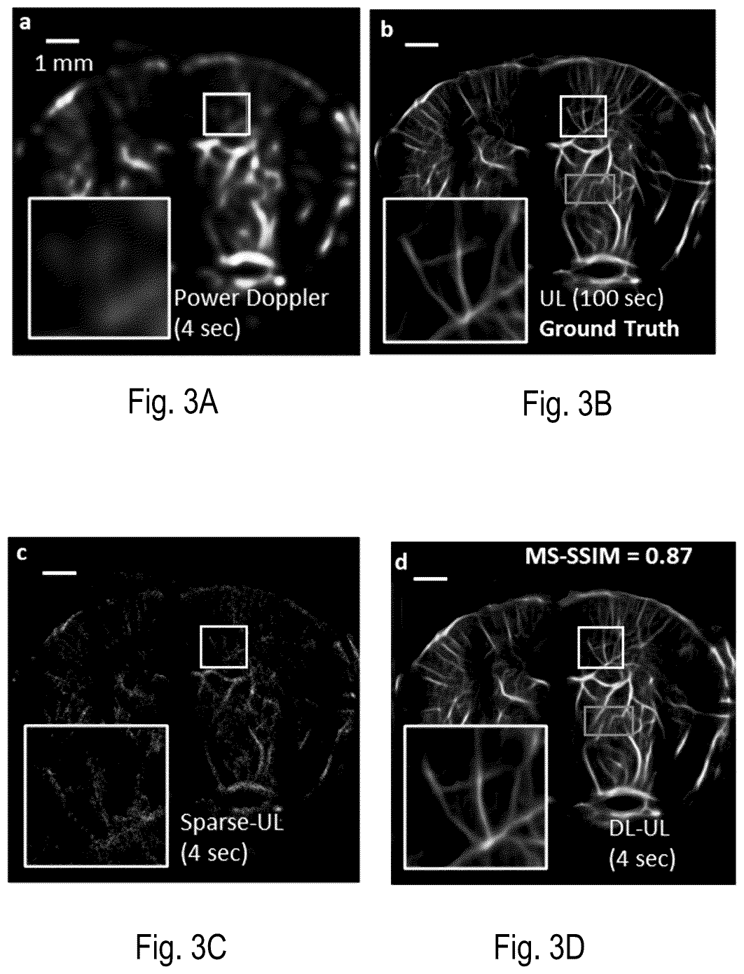

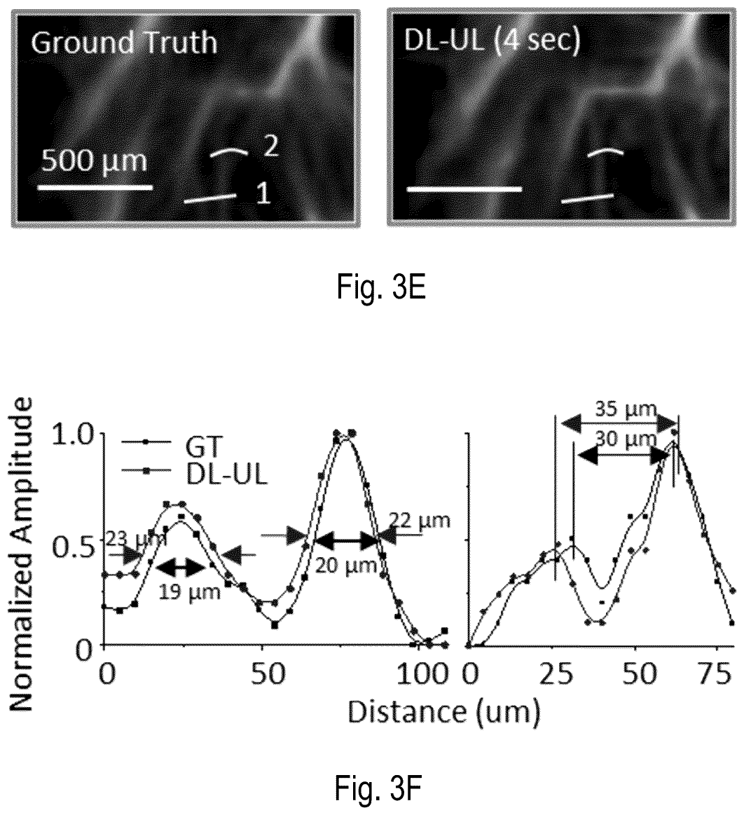

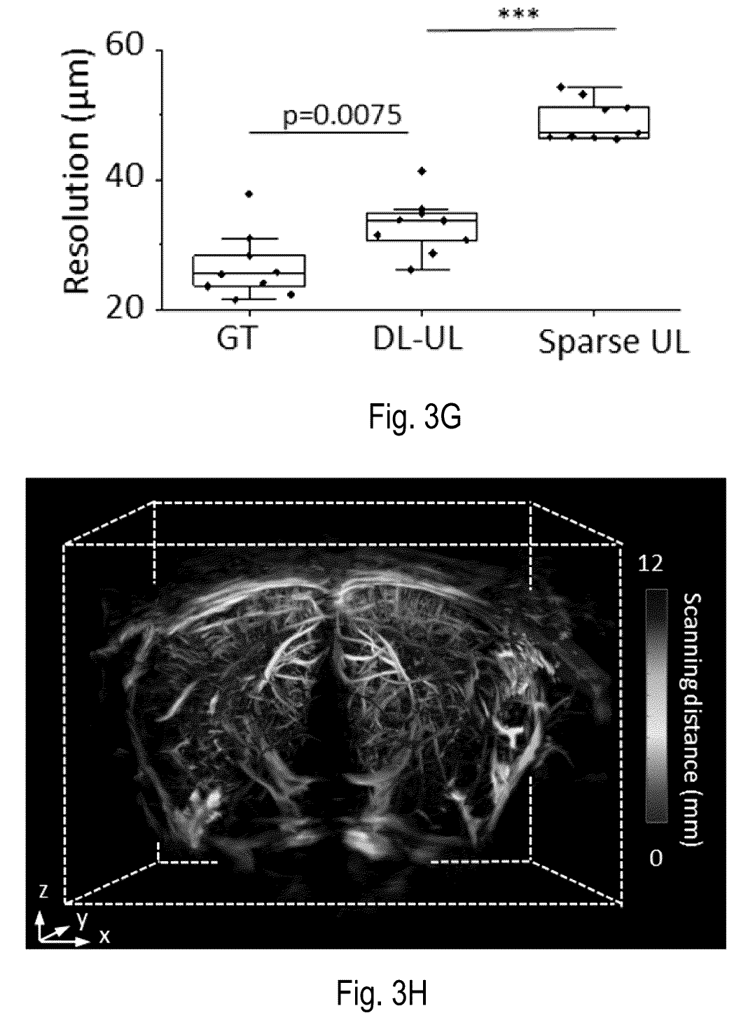

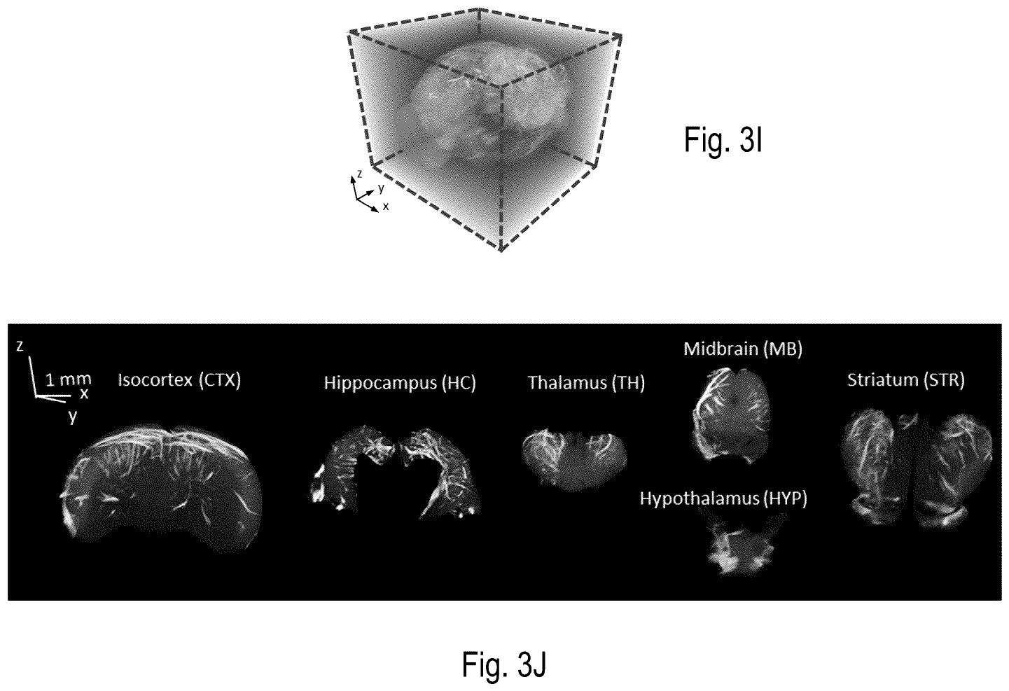

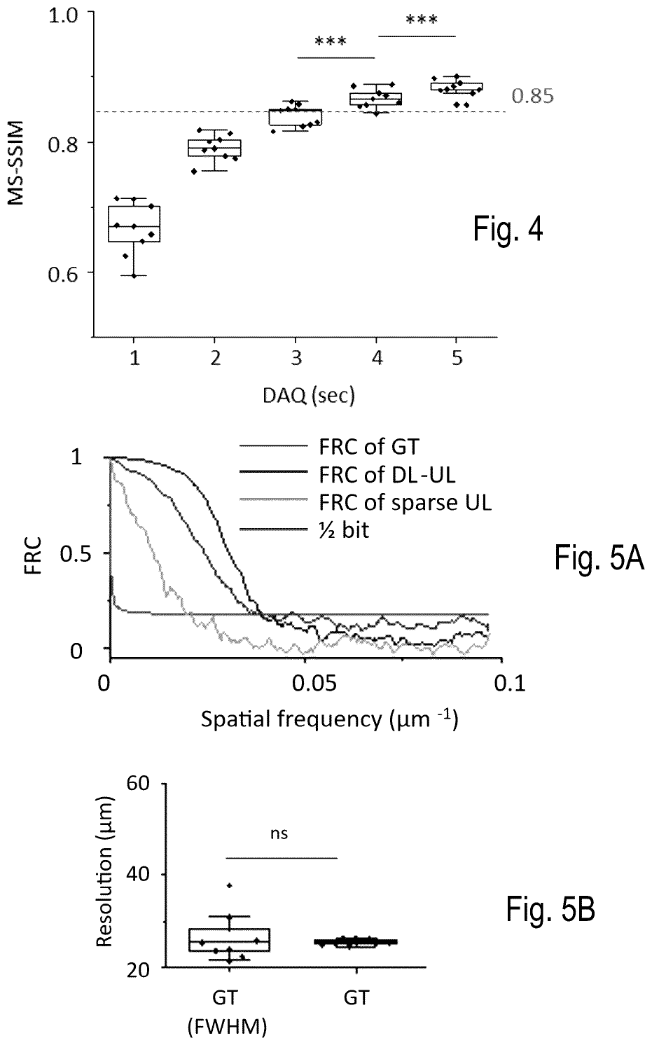

FIG. 3A to FIG. 3D showcase the significant acceleration of the deep learning UL (DL-UL) in a mouse brain image. The power Doppler image (FIG. 3A and conventional UL images (FIG. 3B) may be compared by imaging the coronal views of a mouse brain near the bregma with the injection of low concentrated microbubbles (1×10⁸ bubbles/mL) within a short 4 seconds of DAQ (2,000 ultrasound images). The power Doppler images, while capturing the cerebrovascular structure, appeared fuzzy and primarily resolved only the larger vessels. Conversely, the UL images exhibited higher spatial resolution but displayed sparse and incomplete microvascular networks due to the incomplete DAQ (FIG. 3C). Conventionally, acquiring a fully saturated UL image of the vessel structure necessitated at least 100 seconds of data accumulation, equating to 50,000 ultrasound frames (FIG. 3B). However, it is demonstrated that even with sparse UL images and power Doppler data acquired in just 4 seconds (FIG. 3A, 3C), the trained DL model can effectively predict and reconstruct a detailed, dense cerebral vascular map. Deep-learning models with input from different DAQ times (FIG. 4) are also tested and the output similarity to the fully saturated UL image are assessed (FIG. 3B) using the multi-scale structural similarity index (MS-SSIM). With MS-SSIM=0.85 as the threshold, we found that 4 seconds is sufficient to generate a detailed vasculature map (MS-SSIM=0.87), as shown in FIG. 3D. This process makes DAQ speed 25 times/frame faster than the conventional UL imaging techniques, efficiently producing detailed, dense images of the cerebrovascular system. It is worth noting again that this acceleration does not require an increase in the concentration of the microbubbles.

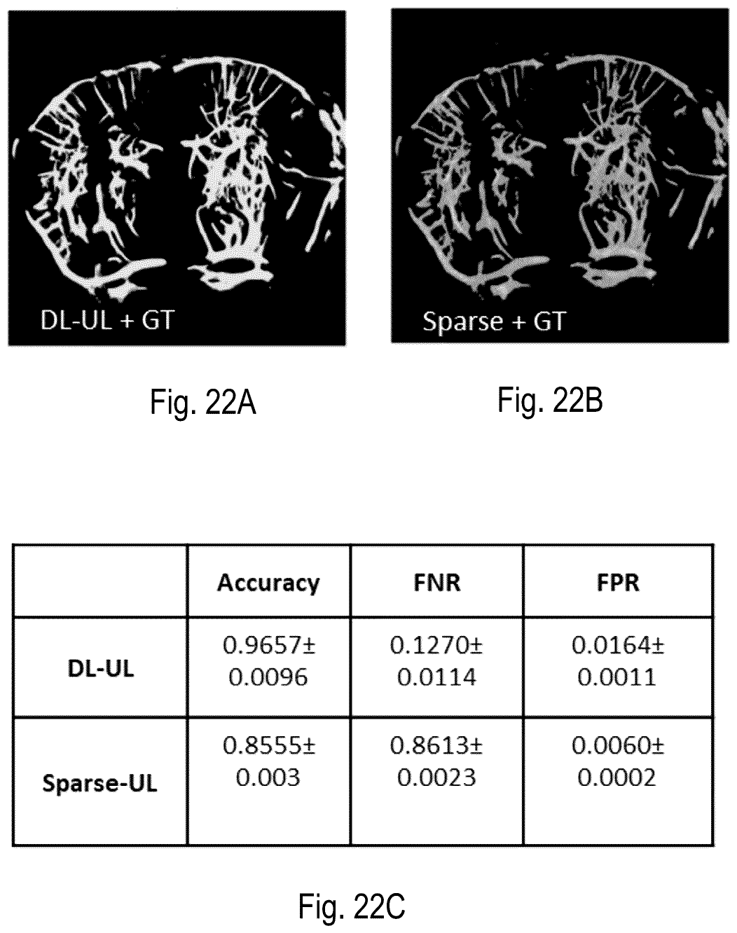

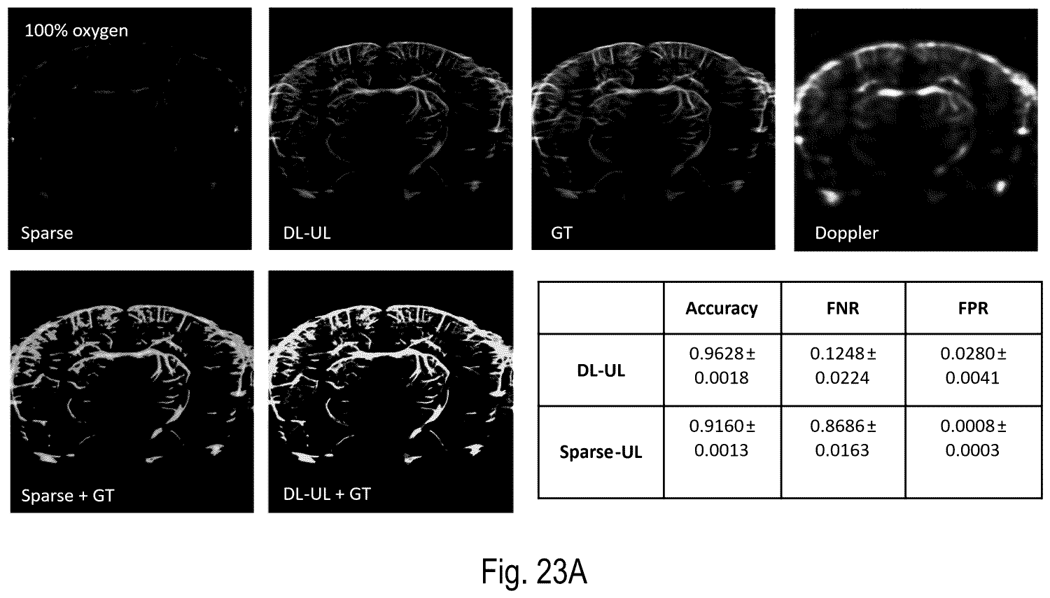

To quantitatively evaluate the DL model, accuracy tests are conducted under different conditions. The ground truth and DL-UL are first converted into the binary image to analyze false positive rate and false negative rate results and then compared them with their respective binary masks. FIG. 22A shows the fusion of DL-UL and ground truth masks, where yellow indicates the overlapped region (true positive); green indicates false negative; and red indicates false positive. For comparison, FIG. 22B displays the fusion of sparse UL and ground truth masks. Sparse UL imaging, which is constructed by a partial dataset, exhibited a significantly lower true positive rate due to incomplete structure caused by insufficient microbubble localization. We calculated accuracy, false negative rate, and false positive rate for both DL-UL and sparse UL quantitatively. As shown in FIG. 22C, both DL-UL and sparse UL have a low false positive rate (0.0164 vs. 0.0006), showing they are effective at minimizing the occurrence of false positive. DL-UL achieved a much lower false negative rate than sparse UL (0.1270 vs. 0.8613), indicating a significantly higher reconstruction rate. DL-UL outperformed sparse UL in accuracy (0.9657 vs. 0.8555). To assess stability, DL-UL performance is evaluated under different blood flow rates induced by different levels of oxygen inhalation (100% and 3%). FIG. 23A-23B visualizes the differences between ground truth, DL-UL, and sparse UL under these conditions. Quantitative results confirmed that DL-UL maintained high accuracy (0.9628 and 0.9670) and low false negative rate (0.1248 and 0.1158) across varying flow rates, demonstrating the robustness of the DL model. Despite the use of a low concentration of microbubbles, the method above maintains high imaging speed while achieving accuracy comparable to other deep learning approaches.

When zooming into a small region, as indicated by blue boxes in FIGS. 3B and 3D, we can observe detailed microvasculature in both the DL-UL and ground truth images (FIG. 3E). To quantitatively assess these microvascular features, amplitude profile measurements are employed. In FIG. 3F, the DL-UL image demonstrates the capability to visualize microvasculature as small as 22 μm and distinguish between two vessels with a gap of 35 μm. In comparison, the full UL image can visualize microvasculature as small as 19 μm and differentiate between two vessels with a separation of 30 μm. The Fourier Ring Correlation method is applied to quantitatively analyze the resolution of sparse UL, ground truth and DL-UL images. Fourier ring correlation assesses the similarity between two independent reconstructions of the same object in frequency space to determine the resolution threshold and the spatial frequency at which both reconstructions are consistent. From measurements (FIG. 3G), it is found that the resolution of the DL-UL images is 24.8±1.6 μm (N=9), which is comparable to the resolution of conventional UL images (ground truth at 25.5±0.6 μm (N=9) with no statistically difference. The 4-second UL images only have a resolution of 49.1±3.2 μm (N=9) due to the sparsity nature of the image. To validate the accuracy of the amplitude profile measurements, the resolutions of ground truth images are compared using both Fourier ring correlation and amplitude profile measurements (FIG. 5A and FIG. 5B). As demonstrated in FIG. 5B, while both methods yielded similar accuracy, the Fourier ring correlation method exhibited a smaller deviation, indicating a greater precision. The resolution of the DL-UL images is quantified and compared to that of conventional UL images (FIG. 3G). From measurements, it is found that the resolution of the DL-UL images is 32.8±4.3 (N=9), compared to 26.6±5.1 μm (N=9) in conventional UL images. The 4-second UL images only have a resolution of 49.1±3.2 (N=9) due to the sparsity nature of the image.

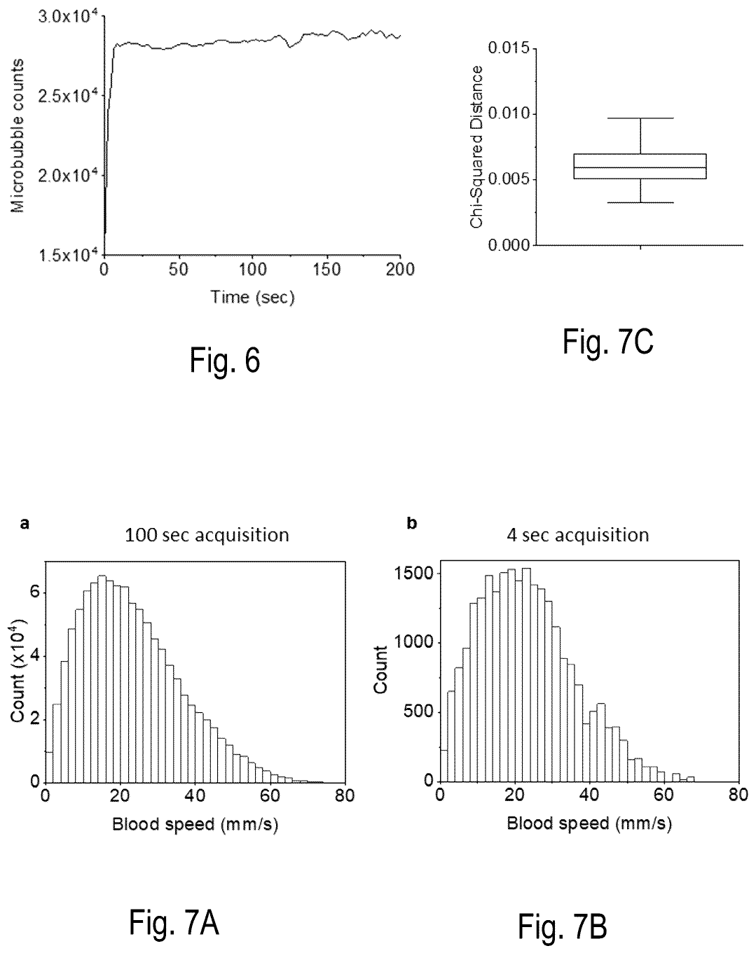

In conventional UL imaging, the maximum allowable dose of the contrast agent caps the overall imaging time, thereby limiting the 3D scanning range with a linear array transducer. Although advanced localization algorithms attempt to accelerate UL imaging by increasing microbubble concentration, the dose ceiling still limits their practical use for large-scale 3D imaging. In contrast, the fast DL-UL imaging facilitates extensive 3D scanning of the entire brain without risking a microbubble overdose. By using a syringe pump to ensure consistent microbubble infusion (FIG. 6), comprehensive brain scanning is achieved with a linear array transducer. FIGS. 3H-3J showcases the whole-brain 3D imaging resulting from this process. The mouse brain is mechanically scanned through, with a step size of 50 μm, covering an overall distance of 12 mm. The collection time is 16 minutes, with a total of 240 frames and 4 seconds of DAQ per frame, which is comparable to the acquisition time (˜15 min) for 3D UL imaging with a matrix array. To put these into perspective, considering conventional UL imaging only has 100 seconds of DAQ per frame, it requires more than 6 hours to reconstruct the complete microvascular map with mechanical scanning, which is not feasible for in vivo imaging. The DL-based localization algorithm is key to overcoming this limitation, reducing the required acquisition time by 25-fold while preserving high-resolution microvascular detail. This dramatic improvement transforms previously infeasible imaging protocols into practical solutions for preclinical applications. FIG. 3H displays the stacked 3D scans of the whole mouse brain in coronal views, color-coded by scanning depth. When registering 3D brain images with Allen Mouse Common Coordinate Framework (CCF), the whole mouse brain microvasculature is presented in FIG. 3I. Further segmentation based on Allen Mouse CCF allows for dividing the whole brain vasculature into six anatomical regions: isocortex (CTX), hippocampus (HPF), thalamus (TH), midbrain (MB), hypothalamus (HY) and striatum (STR). This offers a comprehensive view of the brain's vascular atlas (FIG. 3J).

### DL-UL Imaging for Detailed Cerebral Hemodynamics in Brain Regions

Blood flow information is pivotal in understanding the intricate patterns of cerebral hemodynamics, revealing how different regions receive blood supply under various physiological conditions. Blood flow parameters are crucial for monitoring the brain's responses to BBB modulation because changes in BBB integrity can influence CBF dynamics, impacting brain function and neurological outcomes. Besides visualizing anatomic cerebrovascular networks, it is further demonstrated that the DL-UL imaging technique is capable of correlating imaging signals with CBF in the same super-resolved resolution.

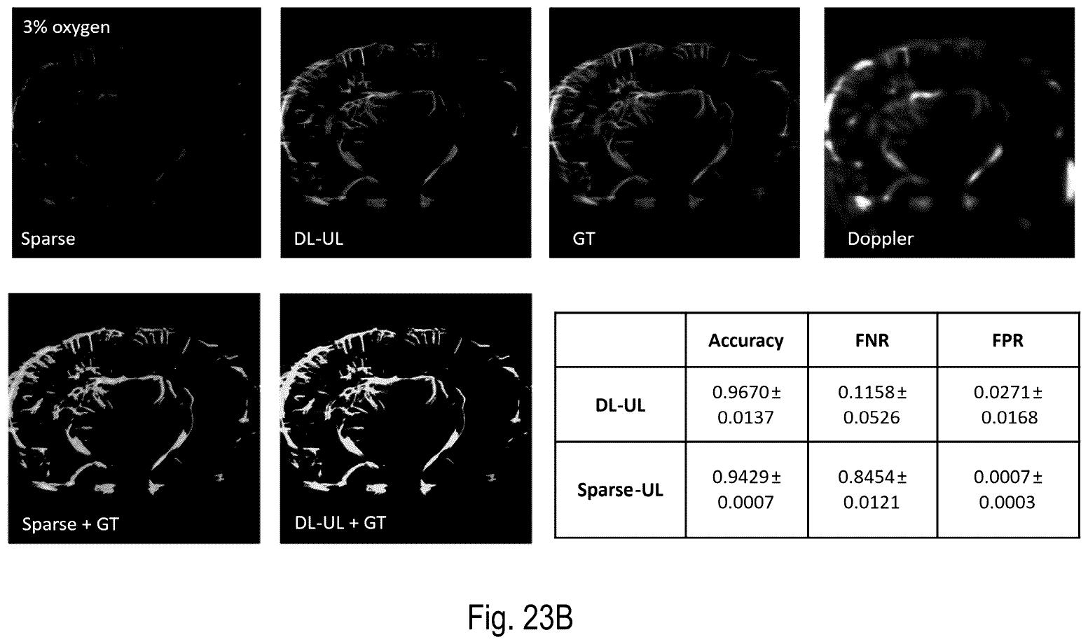

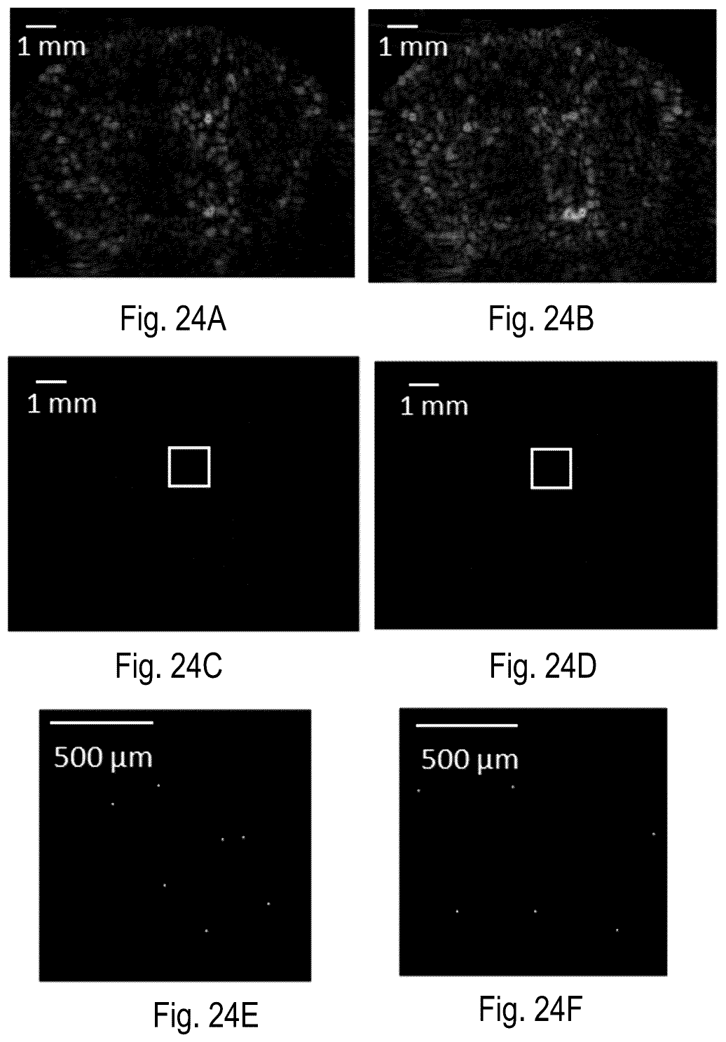

Using UL imaging to map the blood velocity of microvasculature has been demonstrated in both clinical and preclinical settings. Since microbubbles move along with the bloodstream, it is assumed that the moving speed of microbubbles is the same as the local blood flow. Regular UL imaging provides blood velocity map by tracking microbubbles movement. Similarly, in our DL-UL framework, we utilize the sparse data (2,000 frames over 4 seconds) to achieve this. Specifically, we extracted the localized microbubble positions from the sparse input and tracked their movement across frames to compute blood flow velocities. Examples of one frame of microbubble positions are shown in FIG. 24A-24F. To reconstruct the blood flow map, all microbubble locations across frames are tracked using the Hungarian Tracking Algorithm, a multi-objective tracking method. This algorithm creates a cost matrix representing the Euclidean distance of all microbubbles from the current frame to the next. It minimizes the total cost of finding the optimal pairs of microbubbles in adjacent frames. The velocities of each microbubble are calculated by differentiating each position in a trajectory according to the time vector and generated a blood flow velocity map.

To investigate the effect of the short recording time (i.e., low sampling points) in estimating velocity from DL-UL images, the blood speed distribution in a mouse brain are analyzed and compared using image data from 100-second DAQ and 4-second DAQ (FIG. 7). Both images display similar velocity distribution, while the amplitude (sample count) of the 100-second DAQ is around 40 times higher than that of the 4-second DAQ. To quantify this observation, the Chi-squared histogram distance calculation is conducted to measure the dissimilarity between two histograms. The Chi-squared distance is 0.0062±0.0016, indicating the minimal difference (dissimilarity) in the blood velocity contributions. The high similarity of these blood speed distributions suggests that the velocity measurement from a 4-second DAQ is sufficient to represent the overall blood velocity distribution.

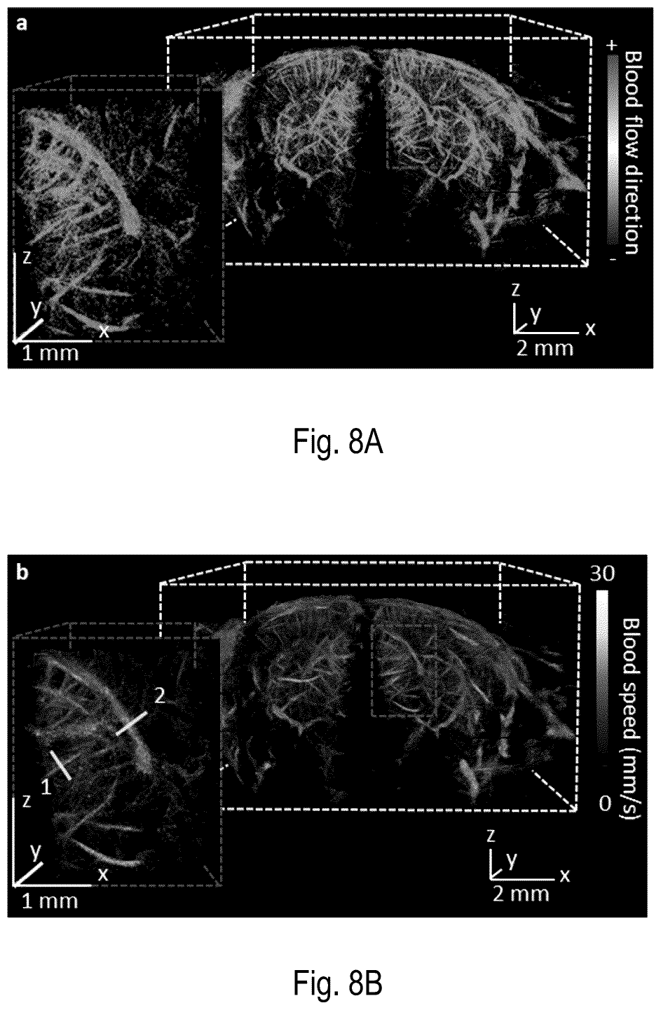

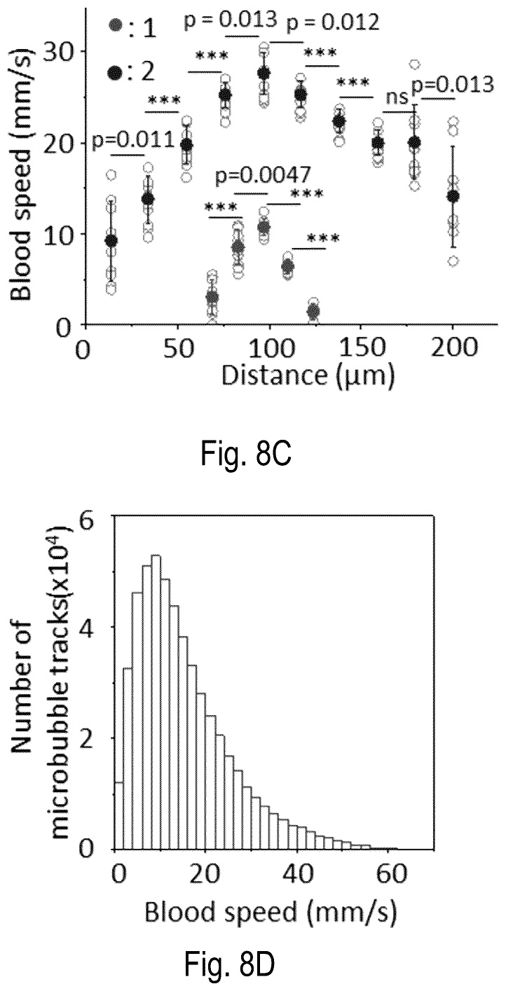

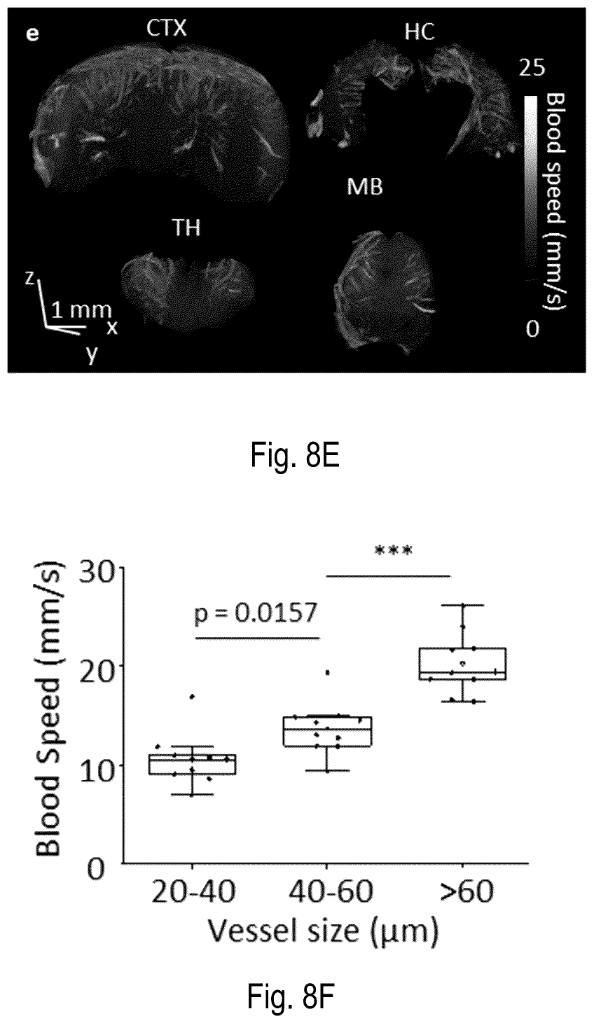

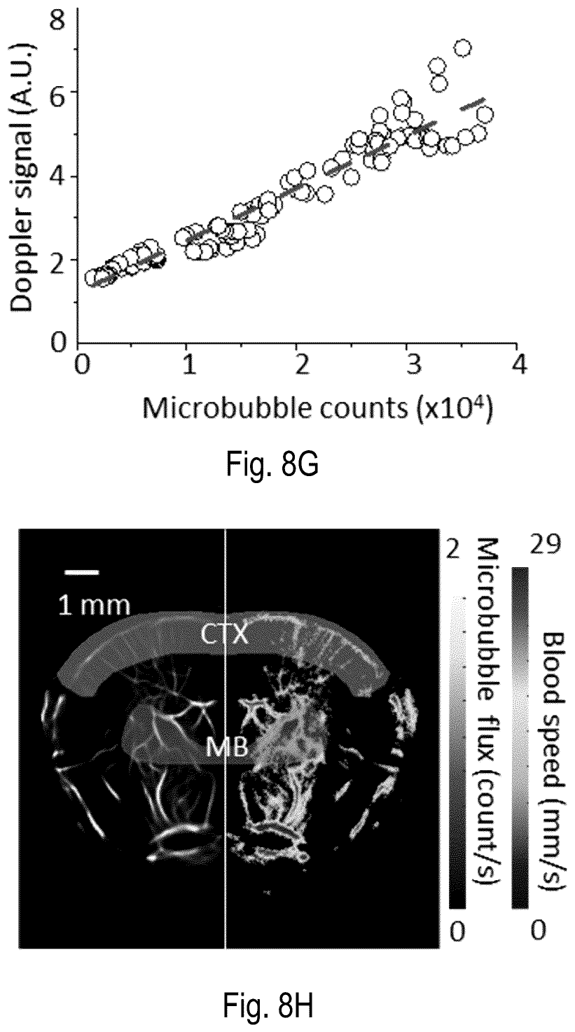

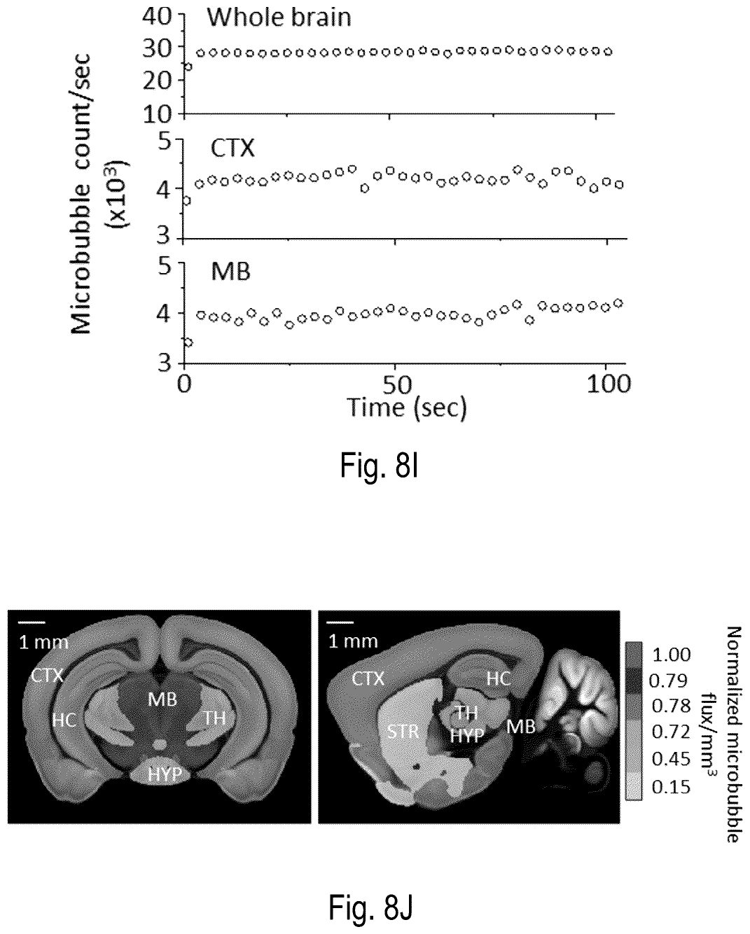

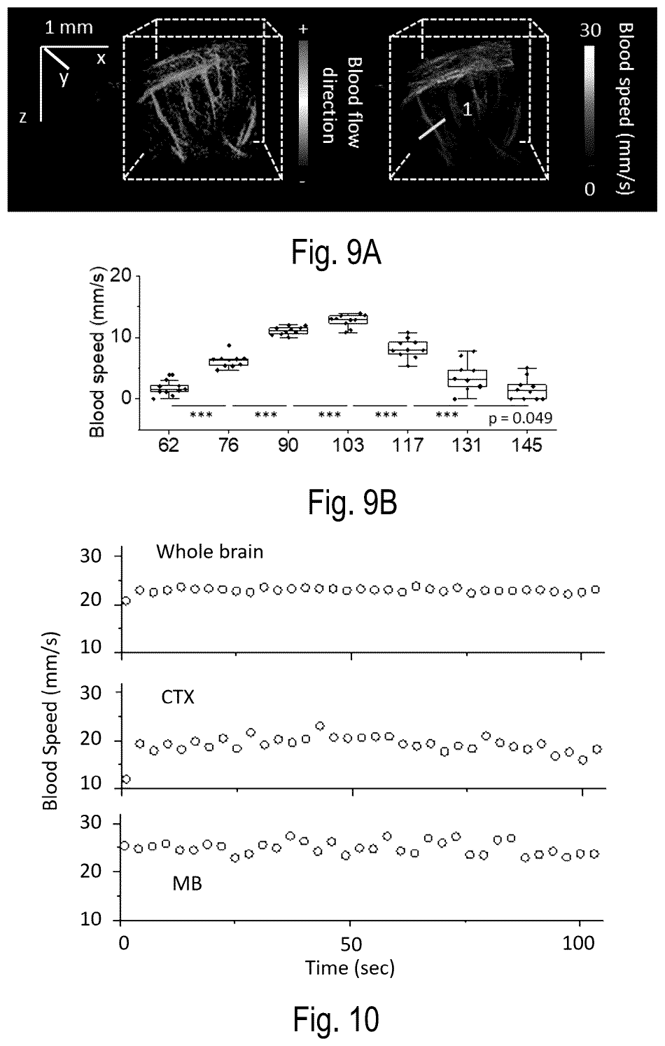

With fast UL blood flow mapping, a 3D coronal view of a mouse brain's blood flow direction map is shown in FIG. 8A and flow speed map is shown in FIG. 8B. The blood flow direction is defined using the standard definition for displaying color Doppler images, where red is the color assigned to flow toward the transducer, and blue is assigned to flow away from the transducer. The super-resolved resolution of blood flow direction and speed maps offer single-vessel resolution into the dynamics of CBF. These maps do not merely show the presence or absence of flow; they quantify the speed in different regions, providing crucial information about brain metabolism and function. Taking advantage of high-imaging resolution, a particularly intriguing aspect of the study is the analysis of the velocity profile in blood vessels. Several blood vessels are randomly selected and the blood speed is analyzed as a function of the distance from the vessel wall. From this analysis, the distribution of blood speed at various positions across the vessels exhibits a Poiseuille-like flow profile (FIG. 8C and FIG. 9A-9B). These observations are crucial as they validate the use of fundamental fluid dynamics principles in the complex environment of cerebral circulation. The relation of blood speed with vessel size (FIG. 8D) is further analyzed. Blood speeds of 10.5±2.5 mm/sec, 13.7±2.6 mm/sec, and 20.3±3.1 mm/sec are measured in vessel size ranges of 20-40 μm, 40-60 μm, and greater than 60 μm, respectively. The method reliably differentiates the ˜3 mm/sec of the blood speed change. Moreover, the high-resolution 3D image allows the segmentation and analysis of blood flow in either a single blood vessel or its anatomical regions. As an example, blood flow mapping of four brain regions (CTX, HPF, TH, and MB) fused with a 3D DL-UL vascular map is shown in FIG. 8E). This segmentation allows for observing variations in vascular structures and flow dynamics across different brain areas, which has implications for understanding region-specific hemodynamic response to BBB modulation.

In addition to blood velocity, another critical parameter for quantifying blood flow is blood flow rate, which measures the volume of blood entering and exiting specific regions of the brain per unit time. Microbubbles, which have been used in tissue perfusion, Doppler, and UL imaging, can serve as hemodynamic indicators to probe blood flow due to their rheology, similar to red blood cells. By assuming that microbubbles move along with the bloodstream, the blood flow rate can be estimated by examining changes in the average number of microbubbles within the region of interest. This assumption is true when maintaining a stable concentration of microbubbles within the bloodstream. To ensure a stable concentration, continuous microbubble injection via the mouse's tail vein may be employed. The microbubble counts are measured over time. As shown in FIG. 6, the result ensures a consistent microbubble concentration in blood for at least 200 seconds, allowing whole-brain 3D scanning.

To confirm the hypothesis that the microbubble signal (number of microbubbles) represents blood perfusion, the power Doppler signal intensity of brain vessels and the microbubble count at the same voxel and duration may be measured. Published results indicate that the power Doppler signal is proportional to changes in blood volume (fractional moving blood volume). Using ultrasound images from the same dataset, we extracted both the power Doppler signals and microbubble counts for comparison. The Doppler signal intensity may be plotted as a function of microbubble count (FIG. 8G). The results show a positive correlation between power Doppler signal intensity and microbubble count, with linear least squares fitting indicating a 0.93 R-squared value. This linear relationship further confirms that fluctuations in microbubble count at each vessel represent the fractional moving blood volume. Additionally, changes in microbubble flux (bubble count/time) are proportional to the relative blood flow rate.

To showcase the multi-parametric capabilities of high-resolution functional UL imaging, both the microbubble flux map and the blood speed map of the same half-brain in a coronal view may be plotted. For better visualization, the speed map may be mirrored and placed on the right side of the microbubble flux map for comparison (FIG. 8H). Microbubble flux and blood speed maps may be further segmented into different brain regions (isocortex and midbrain), enabling region-specific average blood flow analysis (FIG. 8I and FIG. 10). It is observed that both the isocortex and midbrain maintain similar levels of microbubble flux (4189±122 bubbles/s in isocortex vs 3965±145 bubbles/s in the midbrain), but the average flow speeds differ (19.1±1.9 mm/s in isocortex and 25.05±1.4 mm/s in midbrain), suggesting regional variations in vessel size distribution and density.

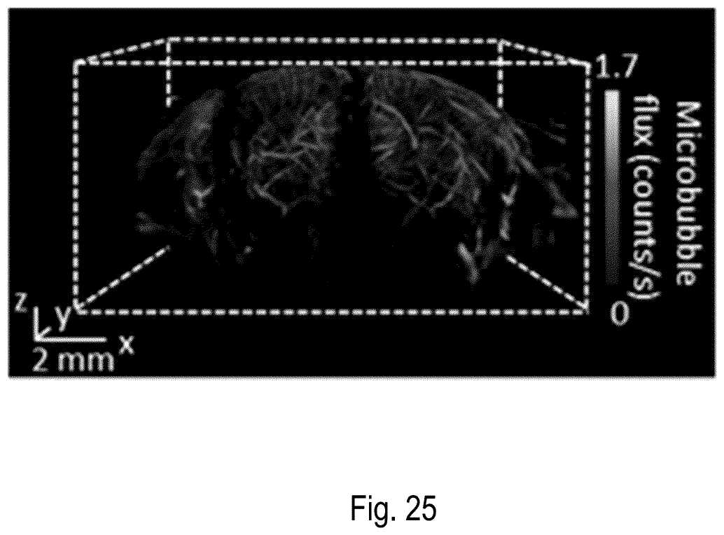

Further, a 3D microbubble flux map (FIG. 25) is registered with the Allen Mouse CCF and calculated the average microbubble flux in different brain regions. The measured flux values are then normalized to the peak value (FIG. 8J), revealing 3D regional variations in microbubble flux. The result shows that the striatum exhibited the lowest flux, while the hippocampus shows the highest flux. These findings align with results obtained from 3D brain UL imaging using a 2D array transducer and show a similar trend to the distribution of regional CBFs obtained from published dynamic Blood Oxygenation Level Dependent MRI (BOLD-MRI) studies. This validation demonstrates the feasibility of using a linear array transducer for 3D functional DL-UL imaging to measure blood flow rates through microbubble flux across the entire mouse brain.

Demonstrate Multi-Parametric Imaging of Brain Blood Oxygenation and Cerebral Hemodynamics with Combined PA and DL-UL Imaging

Blood oxygenation levels are closely linked to the cerebral metabolic rate; areas with higher metabolic activity typically require more oxygen, influencing cerebral blood flow (CBF) and impacting the blood-brain barrier (BBB) integrity. Understanding these dynamics through imaging that assesses blood oxygenation is critical for deciphering the interplay between CBF, metabolic demands, and BBB integrity. Additionally, an imaging technique capable of tracking the diffusion of the BBB permeability tracers is required to monitor the transient permeability increment.

Photoacoustic (PA) imaging is pivotal for non-invasive brain blood oxygenation measurement. It maps oxygenation levels using the distinct spectral responses of oxyhemoglobin and deoxyhemoglobin without additional labeling. PA tomography, a deep-tissue PA imaging type, uses diffused photons to generate acoustic signals captured by an ultrasound array, penetrating a few centimeters into tissue and creating whole-brain oxygenation maps in small animals. PA's optical absorption sensitivity also tracks dye-based BBB permeability tracers. While PA tomography measures blood flow, it is less sensitive and has lower resolution than Doppler ultrasound and UL imaging, which cannot image blood oxygenation or BBB permeability tracers. Combining PA and UL provides complementary imaging capabilities for comprehensively monitoring BBB opening processes.

To integrate PA and DL-UL imaging techniques, the laser is synchronized with the ultrasound system to create an integrated acquisition sequence. The laser generates a pulse and sends a trigger signal to the ultrasound system, which then records the PA signal. Subsequently, the system alternates between transmitting and receiving multiple ultrasound signals over 100 milliseconds, with each ultrasound frame captured at a pulse repetition frequency of 500 Hz. This setup achieves a 10 Hz frame rate for PAUL imaging, matching the PA imaging frame rate and enhancing the visualization of the cerebral microvasculature. Compared with other PAUL imaging framework with linear arrays, the DL enhanced PAUL achieved both high acquisition speed and high reconstruction speed without requiring high concentrations of microbubbles

To validate the imaging capability, an in vivo oxygen challenge study designed to probe key parameters of cerebral hemodynamics under varying conditions of oxygenation inhalation is conducted. Initially, subjects inhaled 100% oxygen for 100 seconds, establishing a hyperoxic baseline for cerebral oxygenation and blood flow. This is followed by 100 seconds of inhaling a hypoxic mix of 3% oxygen and 97% nitrogen, creating a physiological challenge that prompted cerebral vasculature responses. These hyperoxia and hypoxia cycles are repeated to observe dynamic changes in microbubble flux (blood flow rate), blood flow speed, and blood oxygenation, recorded by the PAUL images throughout the process.

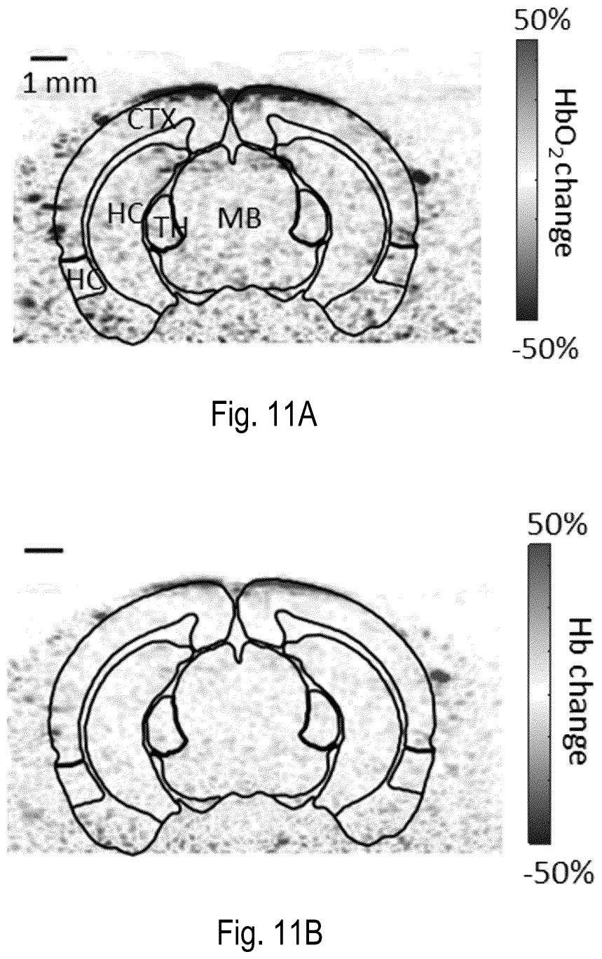

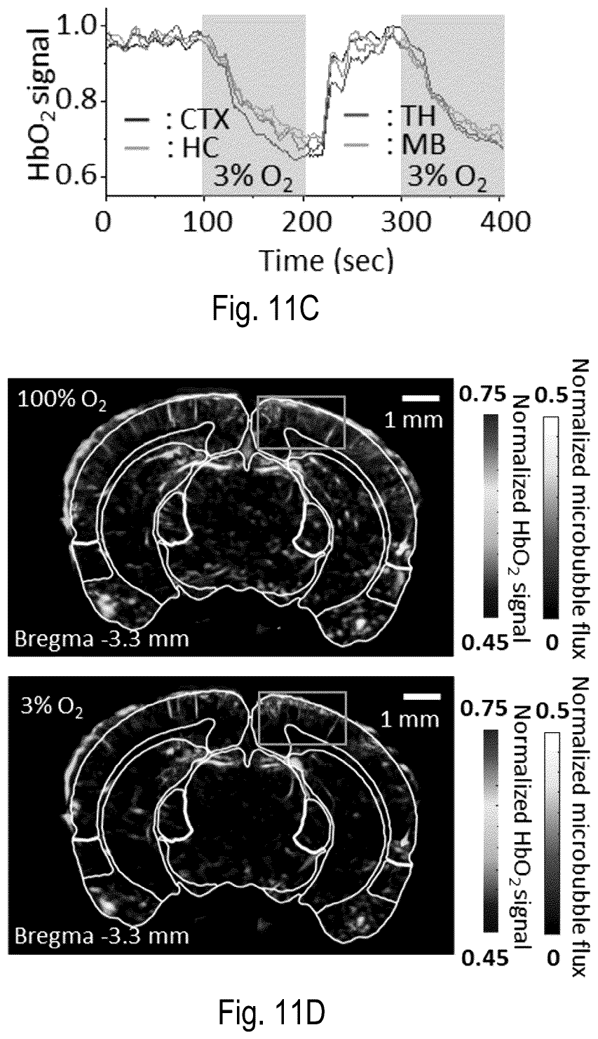

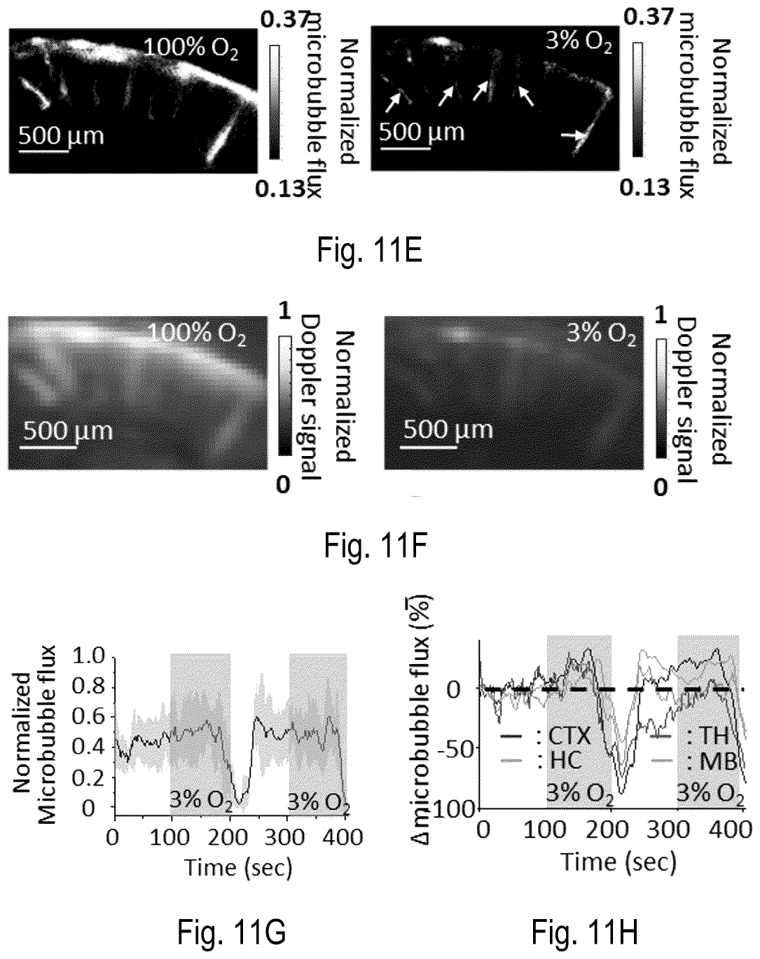

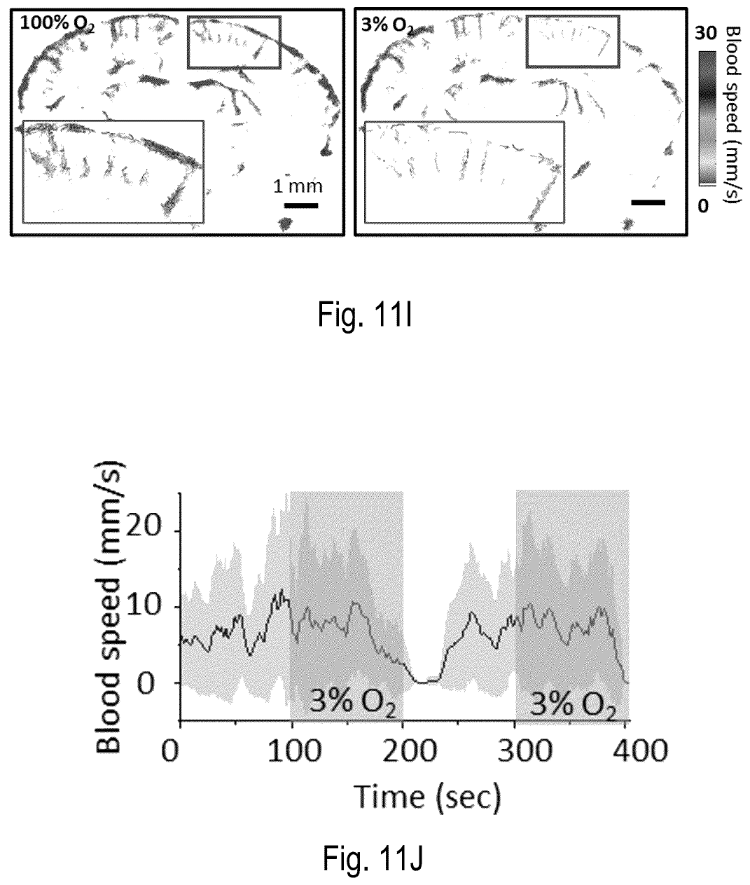

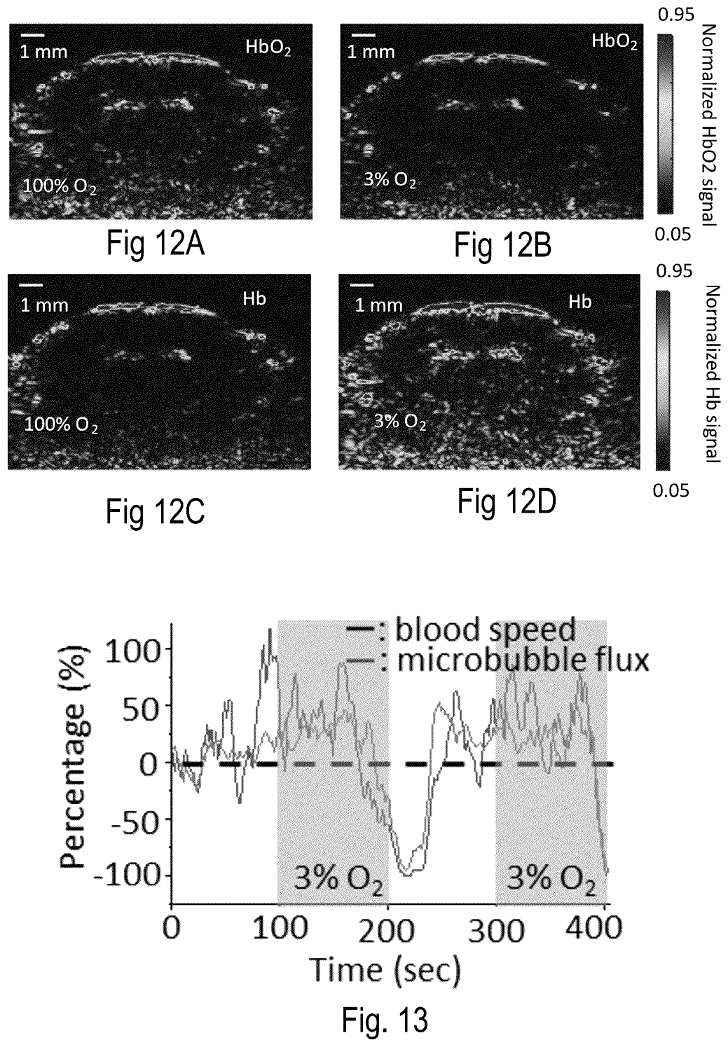

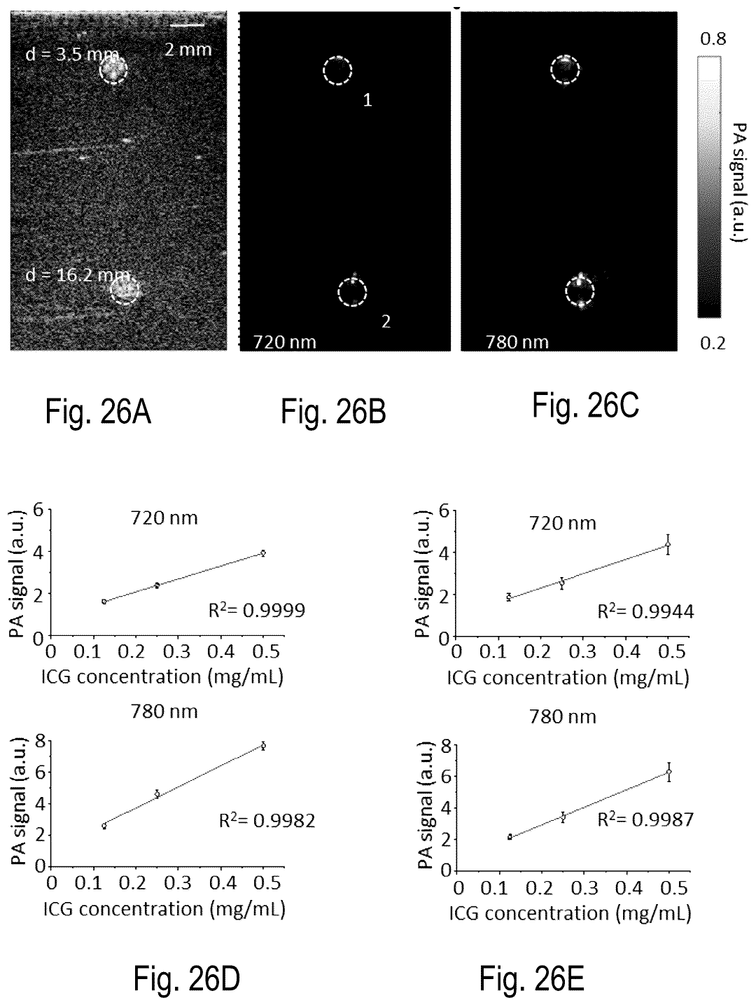

As demonstrated in FIG. 11A-B (and FIG. 12), dual-wavelength PA imaging (750 nm and 850 nm) is used to effectively differentiate between oxygenated (HbO2) and deoxygenated (Hb) hemoglobin states through linear spectral unmixing, and mapped the HbO2 and Hb of the whole brain. While the use of a linear array introduces limited-view artifacts, these artifacts primarily affect structural accuracy rather than the measurement of relative signal changes. To validate this, we conducted a tissue phantom experiment (FIG. 26A-26E), demonstrating that PA signal intensity maintains a strong linear correlation with absorb concentration despite the limited-view effect. This confirms that relative changes in PA signals can be reliably quantified. Leveraging this robustness, HbO2 levels in the isocortex, hippocampus, midbrain, and striatum regions from PA images over two hyperoxia and hypoxia cycles are analyzed. The result shows that the HbO2 levels in each region gradually decreased by about 40% within 100 seconds when the inhalation oxygen levels switched from 100% to 3% (FIG. 11C). However, the HbO2 levels recovered faster when the inhalation oxygen levels are switched back. Similar HbO2 kinetic behavior is observed in clinical data acquired by near-infrared spectroscopy

The relationship between hemodynamics and blood oxygenation is further investigated with multi-parametric PAUL imaging. As shown in FIG. 11D, the dual-contrast images provide a co-registered view of cerebral blood oxygenation and microbubble flux changes in the cerebral microvasculature during 100% oxygen inhalation (top) and 3% oxygen inhalation (bottom). Zoomed-in images (FIG. 11E) highlight a region selected by a rectangle in FIG. 11D, showcasing the super-resolved functional imaging capability in visualizing microbubble flux changes in each cerebral micro-vessel between two oxygen states. The bright images of individual vessels indicate increased blood flux during hyperoxia. For comparison, conventional power Doppler images of the same location (FIG. 11F) are also presented. While power Doppler confirms increased blood flow in hyperoxia and reduced flow in hypoxia, it cannot resolve individual vessels.

The quantitative examination of microbubble fluxes in five vessels (marked by arrows in FIG. 11E) in isocortex over two hyperoxia-and-hypoxia cycles reveals time-dependent fluctuations in microbubble flux (FIG. 11G). A slow increase of average microbubble flux rate for 8% (from 0.50±0.21 to 0.58±0.17) in the first 60 seconds and a quick drop in flux rate for 21% (from 0.58±0.17 to 0.27±0.21) tile the end of the hypoxia period are observed. When the inhalation switched back to hyperoxia, an additional 19% reduction (from 0.27±0.21 to 0.02±0.02) of the flux continued for another 16 seconds before the flux graduate increased back.

To investigate the heterogeneity of blood flow across the brain, vessels are randomly selected in four anatomical regions (isocortex, hippocampus, thalamus, and midbrain) and plotted the percentage change of microbubble flux over two hyperoxia-and-hypoxia cycles, as shown in FIG. 11H. In contrast to blood oxygenation, which is relatively uniform across the brain, the microbubble fluxes in different regions show large variations. While similar trends of slow elevation and quick drop are observed in microbubble flux across these regions, the maximum reduction of microbubble flux varies. Data analysis shows that the flux drop is 87.9% in the isocortex, 64.5% in the hippocampus, 74.1% in the thalamus, and 45.5% in the midbrain, but the minimums of the flux reduction occurred at the same time, 215 seconds after oxygen challenging, for all four regions.

Additionally, mapping whole-brain blood flow speed distributions during oxygen-challenging tests using PAUL imaging is explored. The speed is observed to change over time in the images recorded. In FIG. 11I, the coronal view of the whole-brain blood flow speed maps recorded at the time points right at the end of the hyperoxia (left panel of FIG. 11I) and hypoxia (right panel of FIG. 11I) inhalation period is shown. Hight imaging resolution allows to zoom-in to a region in the isocortex of FIG. 11I to focus on a few blood vessels. The blood speeds in the same vessels indicated in FIG. 11E are quantitatively analyzed over 400 seconds (FIG. 11J). A similar dynamic behavior to that in microbubble flux measurements is observed. To compare the difference between microbubble flux and blood speed measurement side-by-side, percentage changes of averaged microbubble flux from FIG. 11G and of averaged blood speed from FIG. 11J over the whole oxygen challenging period (FIG. 13) are calculated. In comparison, the average blood speed fluctuated more than the microbubble flux due to the tracking errors. Both parameters show a similar trend, the same percentage of reduction, and the minimum occurs at the same time.

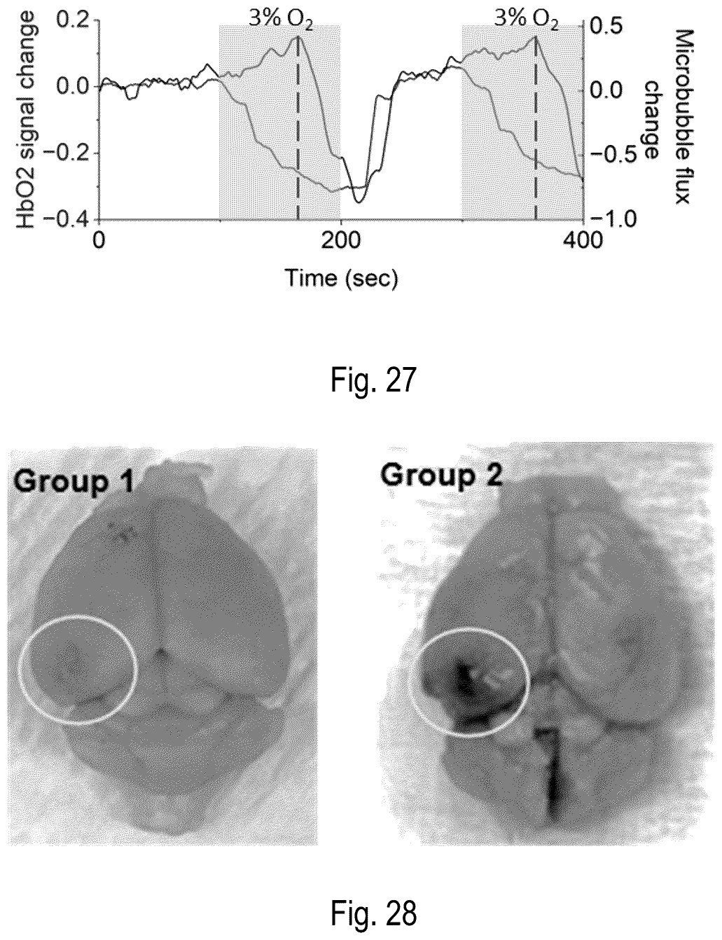

As discussed earlier, microbubble flux is linearly proportional to blood flow rate. Variations in blood oxygenation affect cerebral vasculature through vasoactivity. Generally, reduced blood oxygen levels, or hypoxia, can lead to vasodilation and increased BBB permeability, while hyperoxia may cause vasoconstriction and decreased BBB permeability. However, this simple mechanism of CBF regulation by blood oxygen levels does not cover all scenarios. While hypoxic environments typically increase CBF, hypoxic conditions at high altitudes, such as severe acute hypoxia, can compress blood vessels and reduce CBF. From the HbO2 measurement (FIG. 11C), a 40% HbO2 drop is observed, which is considered a much more severe acute hypoxia condition than the hypoxia conditions tested in clinical studies. It may be attributed to the observed complex dynamics of the microbubble flux and blood speed reduction. To provide further clarity, in FIG. 27, the changes in tissue oxygenation and microbubble flux during the oxygen challenge are overlaid. Initially, as 3% oxygen was introduced, tissue oxygenation gradually decreased while microbubble flux increased, consistent with vasodilation induced by hypoxia. However, under acute hypoxic conditions (3% oxygen), blood flow did not continuously increase. About 63 seconds after introducing 3% oxygen, when tissue oxygenation dropped below 30%, A significant reduction in microbubble flux is observed. This likely reflects the vasculature's response to severe oxygen deprivation. This pattern was consistent across two cycles of the oxygen challenge, demonstrating the reliability of our imaging system. These observed dynamics underscore the complexity of cerebral vascular responses under acute hypoxia and highlight our approach's ability to capture these nuanced changes

### Monitoring Cerebral Hemodynamic Changes Induced by FUS-Mediated BBB Disruption Using Whole-Brain PAUL Imaging

FUS-mediated BBB opening has shown promise as a non-invasive method for facilitating drug delivery to treat brain health conditions such as neurodegenerative diseases and brain tumors. FUS transiently increases BBB permeability by oscillating injected microbubbles to exert mechanical forces on the endothelial cells lining the blood vessels. In the disclosure herein, it is demonstrated that DL enhanced PAUL imaging can monitor the vascular response caused by FUS-mediated BBB disruption and simultaneously track the diffusion of tracers across the BBB throughout the entire brain.

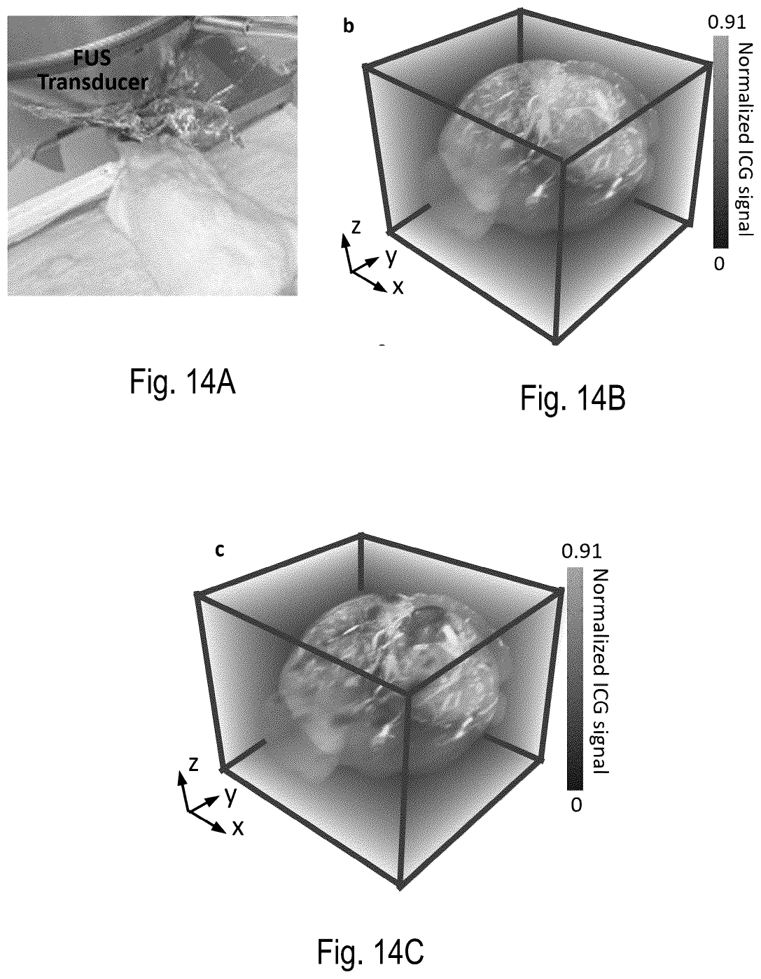

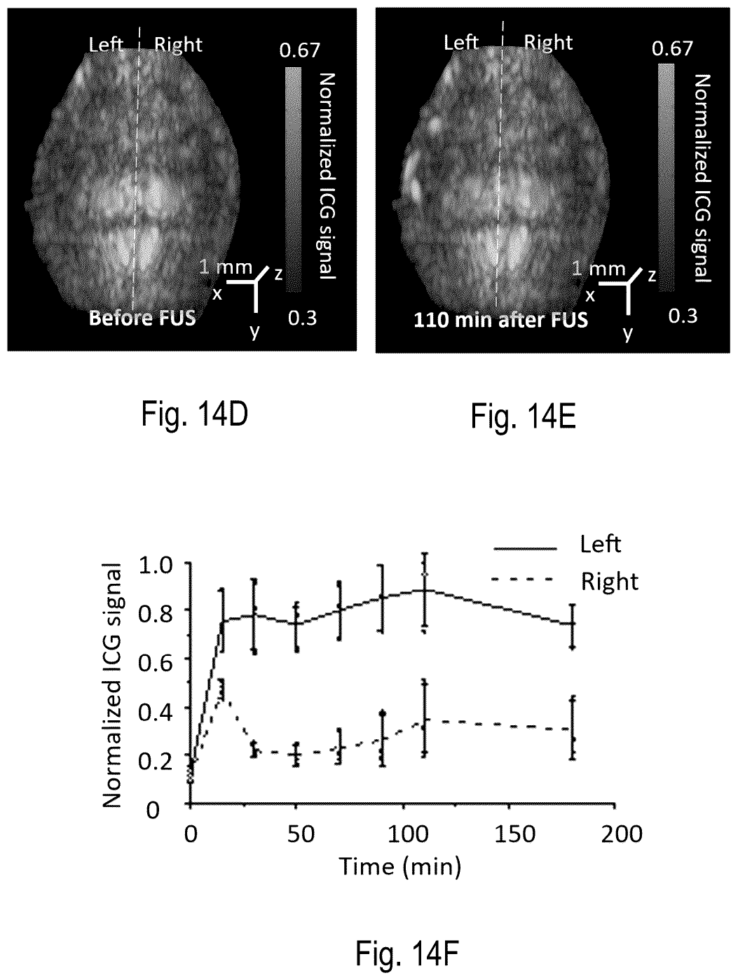

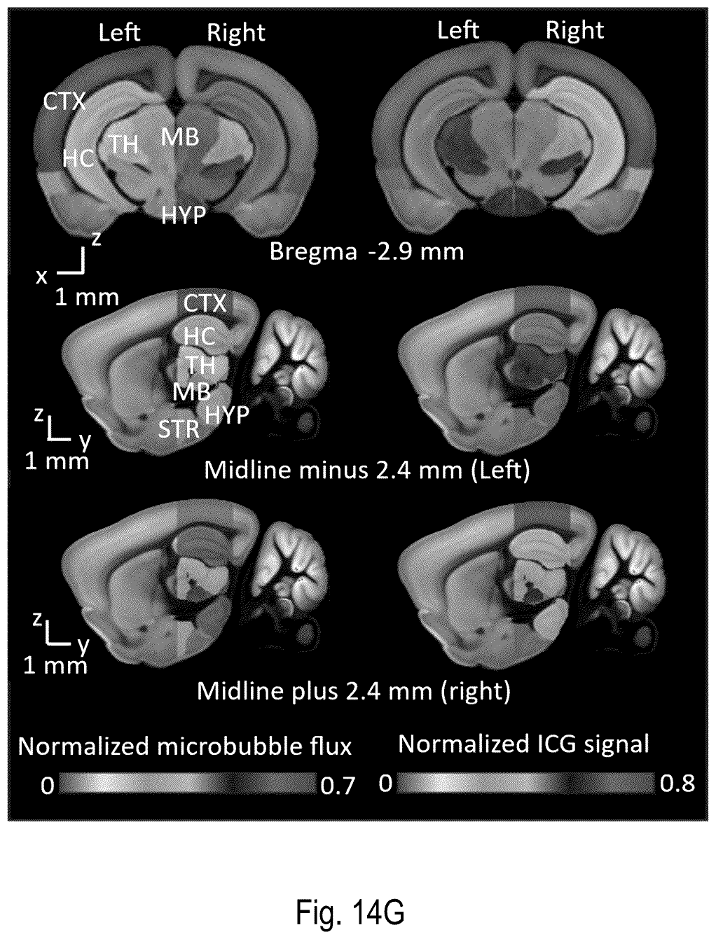

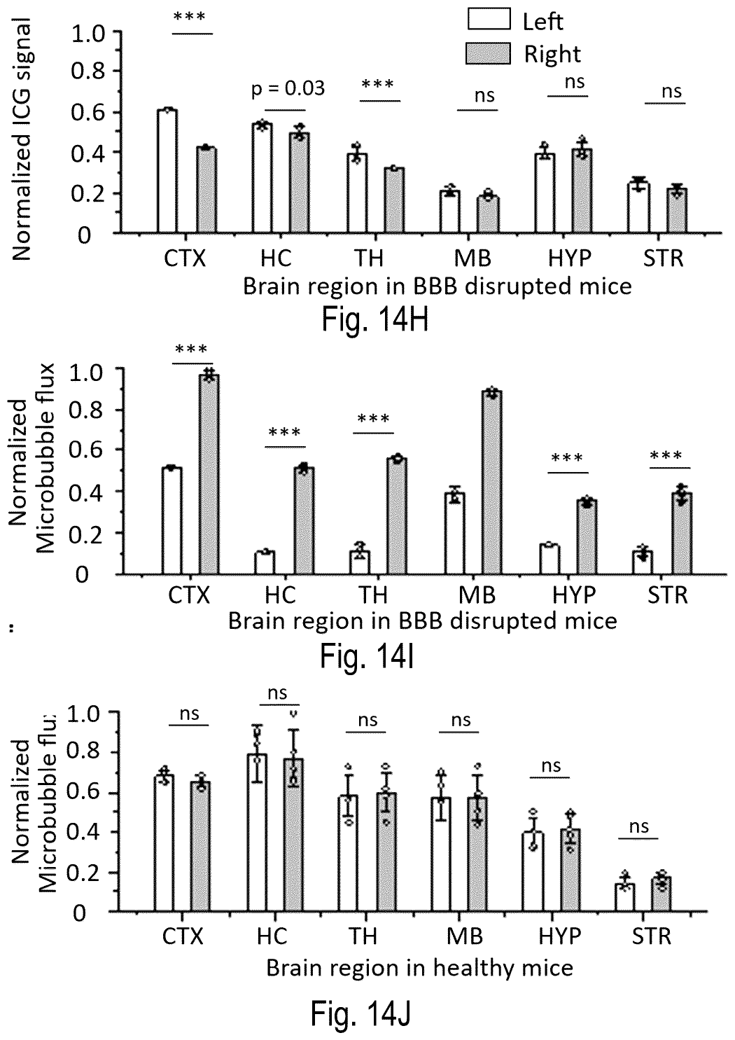

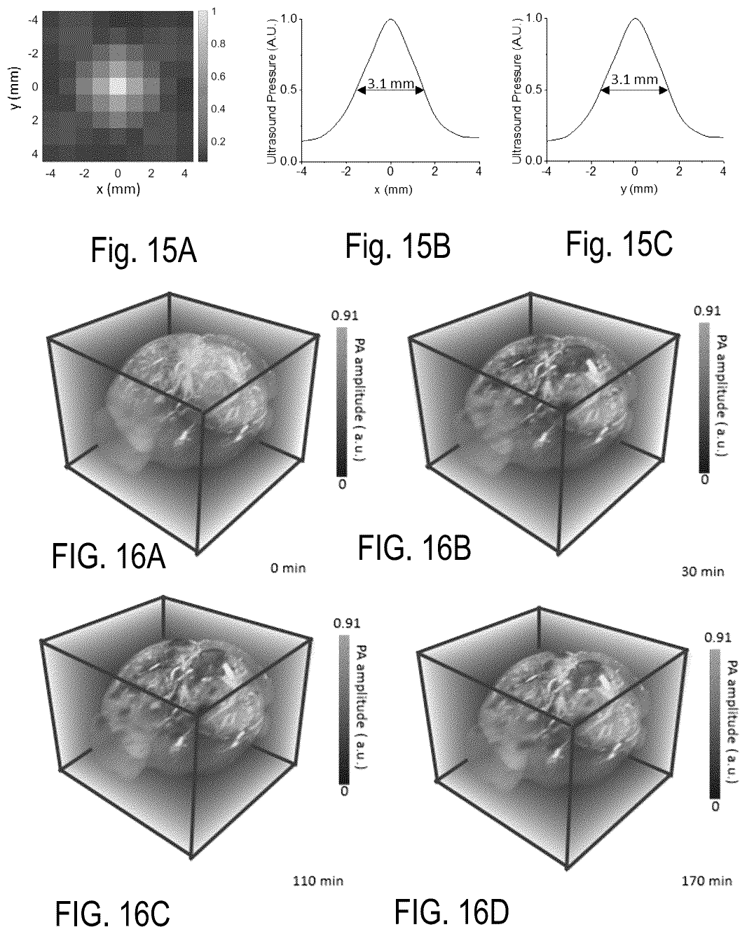

FUS-mediated BBB disruption is first applied on the left hemisphere of a mouse brain before imaging (FIG. 14A) with a 0.5 MHz HIFU single-element transducer. The focal spot of the transducer is 3.1 mm in radius at the peak intensity, characterized by the FWHM of the ultrasound pressure (FIGS. 15A-15C).

To achieve safe BBB disruption without causing vascular damage, the FUS parameters are optimized by adjusting the total exposure time while keeping other parameters constant. Specifically, we maintained a peak pressure of 0.2 MPa, a burst length of 2 msec, and a repetition rate of 1 Hz and injected 50 μL of microbubble solutions (1×10⁸ bubbles) during activation. The resulting mechanical index (MI) of 0.27 falls within the clinically safe range (MI=0.2-0.6). Two representative cases were examined: Group 1 received 30 seconds of FUS exposure, while Group 2 was treated with a fourfold increased exposure (a total of 120 seconds, delivered as two 60-second on/off cycles). Untreated healthy mice served as the control group.

Following FUS treatment, 100 L of Evans Blue solution (18 mg/mL) was injected to visualize changes in BBB permeability, due to its established use and easy visualization by photography. After a two-hour circulation period to allow for dye clearance, the mice were sacrificed, and their brains were extracted following transcardiac perfusion with PBS. FIG. 28 displays the distribution of Evans Blue in representative brain tissue from both groups. While both groups showed evidence of BBB opening through dye retention, Group 2 exhibited a darker Evans Blue signal accompanied by visible blood, indicating vascular and tissue damage. In contrast, Group 1 did not show any visible bleeding.

Histology analysis is further conducted by preparing coronal sections (5 μm thick) of brain tissue and stained with hematoxylin and eosin (H&E) and TUNEL to assess hemorrhage and neuronal apoptosis, respectively. FIG. 29A-29B shows the H&E stain of the brain slices selected from Group 1 and Group 2. To evaluate whether hemorrhage occurred caused by FUS blood vessel damage, we zoomed the selected positions to look for signs of red blood cell extravasation in perivascular areas. Three regions are selected from both brains at different depths along the FUS beam path. Three regions are also selected at similar depths from the right brain of the top image for comparison. Those positions are labeled as Position 1-9, as shown in FIG. 29a, and presented the high-magnification microscopic images of these positions in FIG. 29b. Images in positions 1-3 show healthy brain parenchyma with no evidence of vascular disruption or hemorrhage; cells and vessels are intact, indicating normal histology. For the low ultrasound energy exposed areas, the image of position 4, near the surface, shows mild structural disruption in the parenchyma. Arrows indicate subtle perivascular changes. However, there is no evidence of hemorrhage or overt damage to vasculature. Like positions 1-3 in control regions, positions 5-6 show normal tissue architecture with no extravasation of red blood cells or other pathological changes. It is worth noting that the foci of the FUS aim at the regions of position 5-6 instead of position 4. In contrast to the low FUS dose group, in position 7-8, where the tissue was exposed to a high dose of FUS, images show the disruption of the parenchymal structure and significant extravasation of red blood cells, where arrows indicate the extravasation of red blood cells. In position 9, similar to position 4, the image shows mild structural disruption in the parenchyma, where arrows indicate subtle perivascular changes. Overall, from the H&E stain, we see that while high-energy FUS causes evident hemorrhagic damage, as seen in histological sections of the exposed hemisphere, low-energy FUS does not induce significant hemorrhage or damage to brain vasculature.

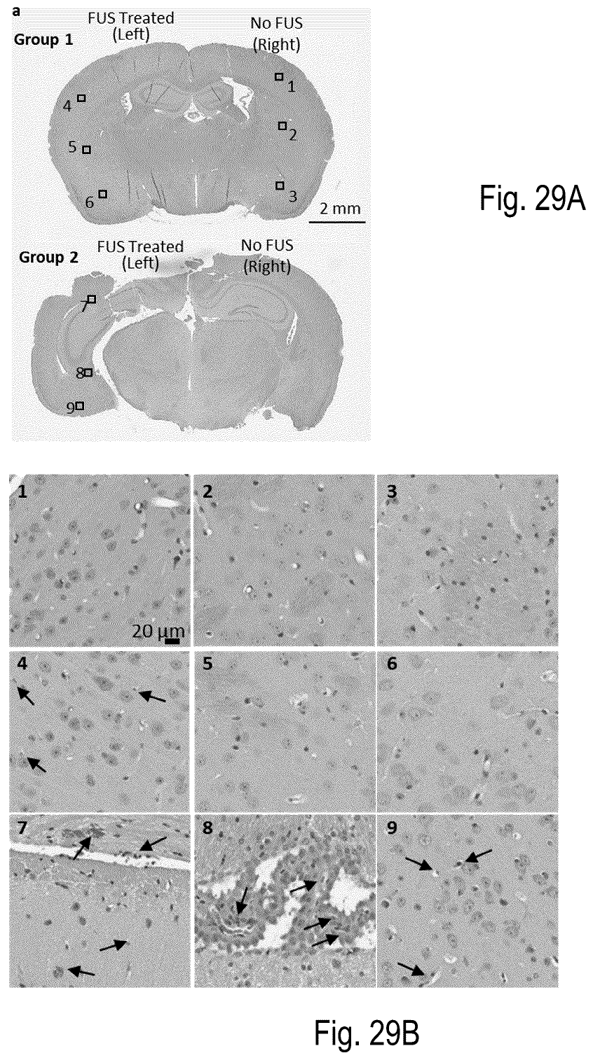

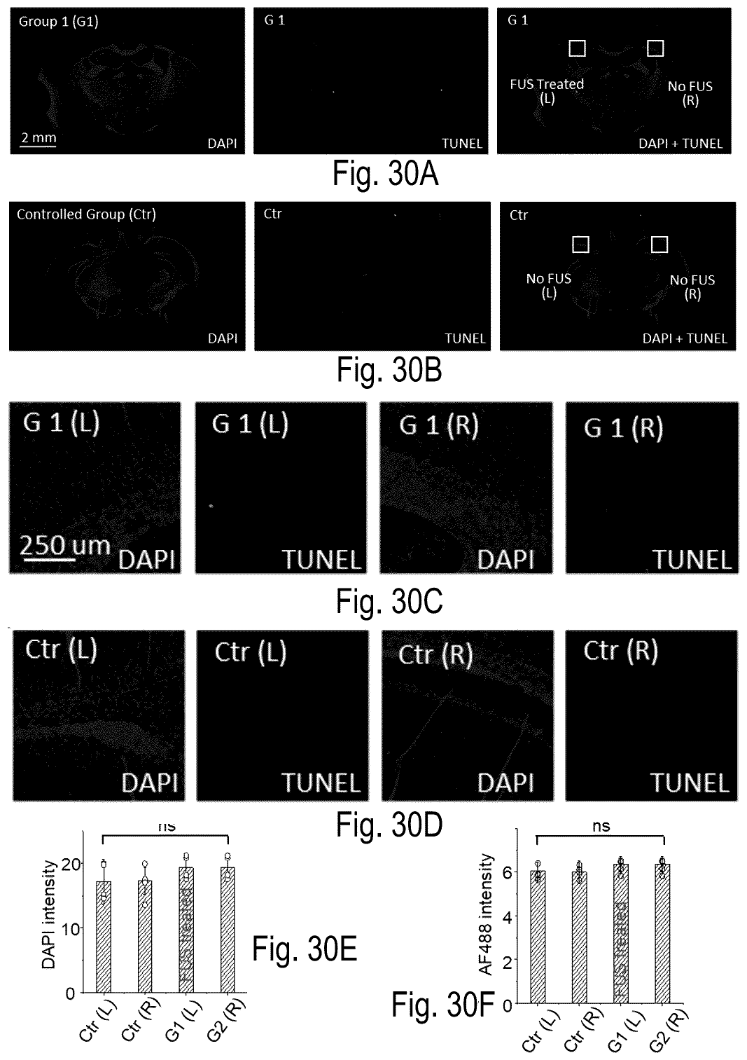

Neuronal apoptosis is further evaluated by performing TUNEL staining. FIG. 30A-3B compares DAPI and TUNEL signals in Group 1 and the untreated Control Group. No strong TUNEL (fluorescence signal) signals were observed in either group. FIG. 30C-30D shows zoomed-in regions of both groups, where the TUNEL signal intensity is similar in both the left and right regions. Quantitative analysis (FIG. 30E-3F) revealed no significant differences in TUNEL and DAPI signal intensity between the groups, indicating that the transient BBB disruption in Group 1 did not induce neuronal apoptosis.

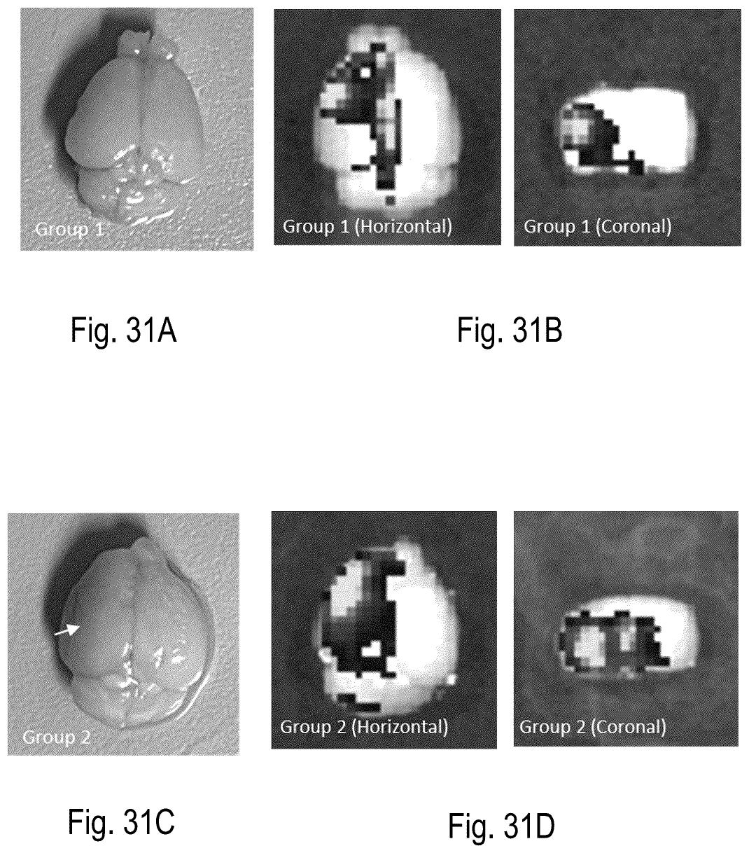

After confirming the safety of the FUS parameters used in Group 1, we applied the same FUS conditions to open the BBB and injected FDA-approved indocyanine green (ICG) as a tracer immediately after FUS irradiation. ICG is selected as a BBB permeability imaging tracer for PAUL imaging because its peak optical absorption is within the NIR tissue window, offering a high ICG signal with a low tissue background. Additionally, the feasibility of using ICG to assess the BBB disruption has been well documented, and ICG retention in the interstitial brain tissue is linked to BBB damage, where the high ICG tissue concentration is limited to the brain areas affected by the BBB disruption. FIG. 31A-31D further validates the ICG tracer mapping.

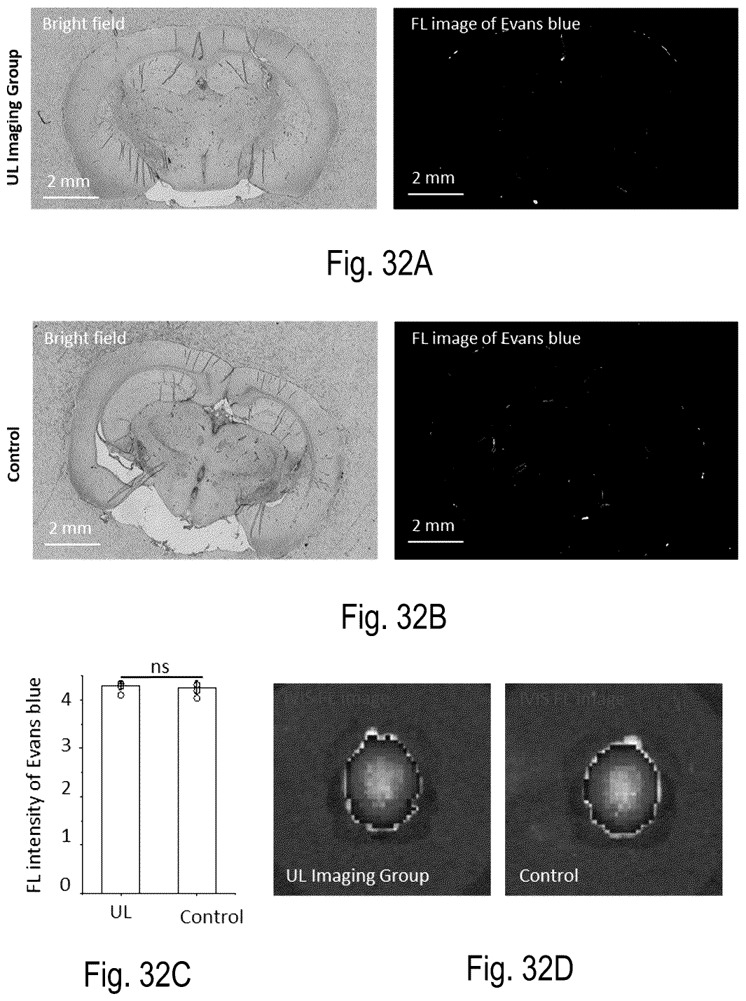

To longitudinally monitor BBB disruption, PAUL imaging sequences are applied before and after ICG solution injection at multiple time points: 15 minutes, 30 minutes, 50 minutes, 70 minutes, 90 minutes, 110 minutes, and 170 minutes. The same concentration of microbubbles (1×10⁸ bubbles/mL) is injected once at 110 minutes for DL-UL imaging. For each time point, the transducer scanned laterally to collect 3D imaging data of the whole brain. Each position underwent 4 seconds of DAQ with laser wavelengths of 700 nm, 780 nm, 800 nm, and 850 nm. The step size is 50 μm for 3D scanning, capturing a total of 260 slices across the brain for 3D rendering. Low-concentration microbubbles (1×10⁸ bubbles/mL) were only injected at 110 minutes for 3D DL-UL imaging with the perfusion rate of 20 μL/min to prevent microbubble overdose. Since ultrasound imaging with microbubbles can potentially induce BBB disruption¹⁰², DL-UL imaging was conducted with an MI of 0.1, which is at least two times lower than the typical therapeutic MI range (0.2-0.6) used for BBB opening. To assess any potential effects, we evaluated BBB permeability after UL imaging with microbubbles and confirmed that BBB integrity remained intact under these conditions (FIG. 32A-32D).

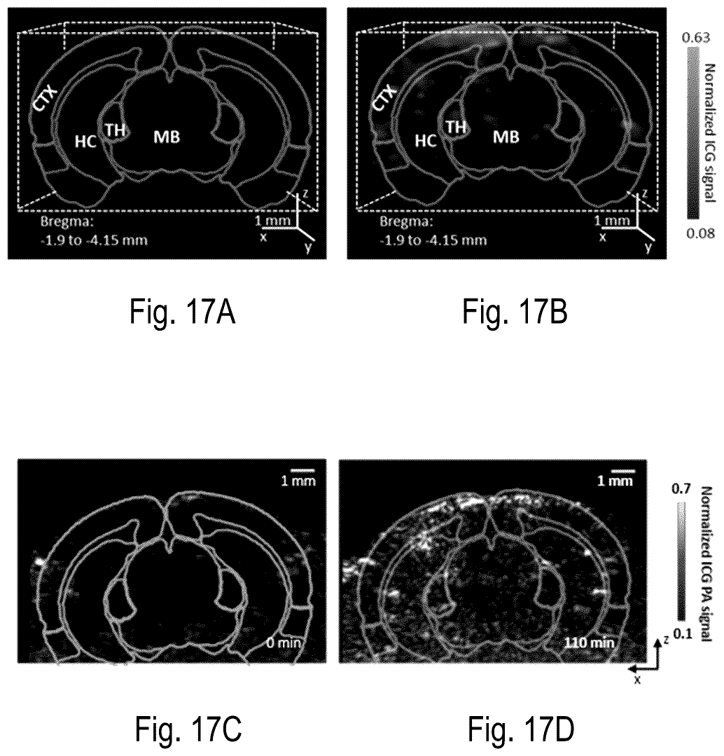

The time-sequenced 3D PA imaging illustrated the diffusion and retention of ICG before BBB opening and 30 minutes, 110 minutes, and 170 minutes post-BBB opening (FIG. 14B-14C and FIG. 16A-16D). To better illustrate the diffusion of the ICG, the transverse and coronal views of the ICG PA images pre and 110 minutes post-BBB opening are also shown (FIG. 14D-14E and FIG. 17A-17D). The light-up region in PA images indicates the increased permeability of BBB, which allows the diffusion and accumulation of the ICG molecules into brain tissue. A quantitative analysis of ICG PA intensities as a function of time on the untreated right brain shows that the ICG signal reaches a peak value roughly 15 minutes after ICG injection (FIG. 14F). The ICG intensity then decreases after 30 minutes of the injection to a level near the baseline. In contrast, at the FUS-treated left brain region, the ICG intensity reaches the maximum intensity and maintains the same intensity for at least 170 minutes during the imaging window (FIG. 14F). The ICG intensity on the left brain is about ˜4 times higher than the untreated left brain 30 minutes after ICG injection, indicating the retention of ICG in interstitial brain tissue.

To investigate the effect of localized BBB permeability change in brain hemodynamic parameters, the change in blood flow rate (microbubble flux) and blood flow speed caused by BBB opening is further investigated. The regional change of vascular parameters in response to the BBB disruption is focused on. To simplify the data analysis, the region is cropped from bregma −1.9 mm to −4.15 mm and exposed to the FUS from the whole brain 3D PAUL image to study the change in vascular functions. To analyze regional change, the blood flow-associated microbubble flux rate is first calculated from the DL-UL signal within each anatomical region and assigned the result to each brain section (Left column of FIG. 14G). Assuming the retained ICG signal is caused by the BBB disruption near blood vessels, to associate the regional microbubble flux change with BBB disruption, the averaged ICG intensity is additional calculated from the PA signal within the corresponding brain sections (Left column of FIG. 14G).

The microbubble flux and the ICG signal in six brain regions are quantitatively analyzed, and the values between FUS-treated left and untreated right brains of the same regions are compared (FIG. 14H-14I). The results show that significant elevation of ICG signal in the left CTX (31.30±1.01%), HC (6.60±3.03%) and TH (17.34±6.59%). In contrast, microbubble flux analysis shows a significant reduction of microbubble flux in left CTX (47.38±1.13%), HC (75.71±1.06%), TH (80.61±5.45%), MB (42.27±2.09%), HYP (61.26±1.71%) and STR (62.91±2.71%). While the ICG distribution is confined within the FUS exposure locally, BBB disruption causes a relatively global impact on the cerebral blood flux despite each region showing different flow responsibility to the disruption. As a comparison, microbubble flux in these brain regions from healthy mice is also evaluated, revealing no difference in microbubble counts between left and right brain regions (FIG. 14J), further indicating the microbubble counts change in FIG. 14I is from BBB disruption.

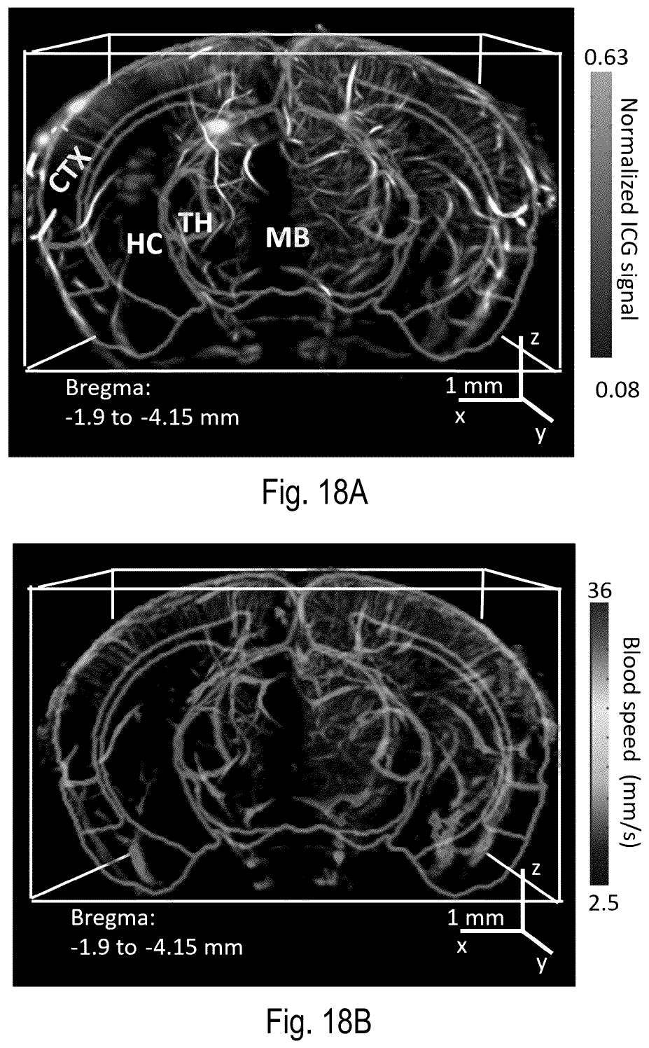

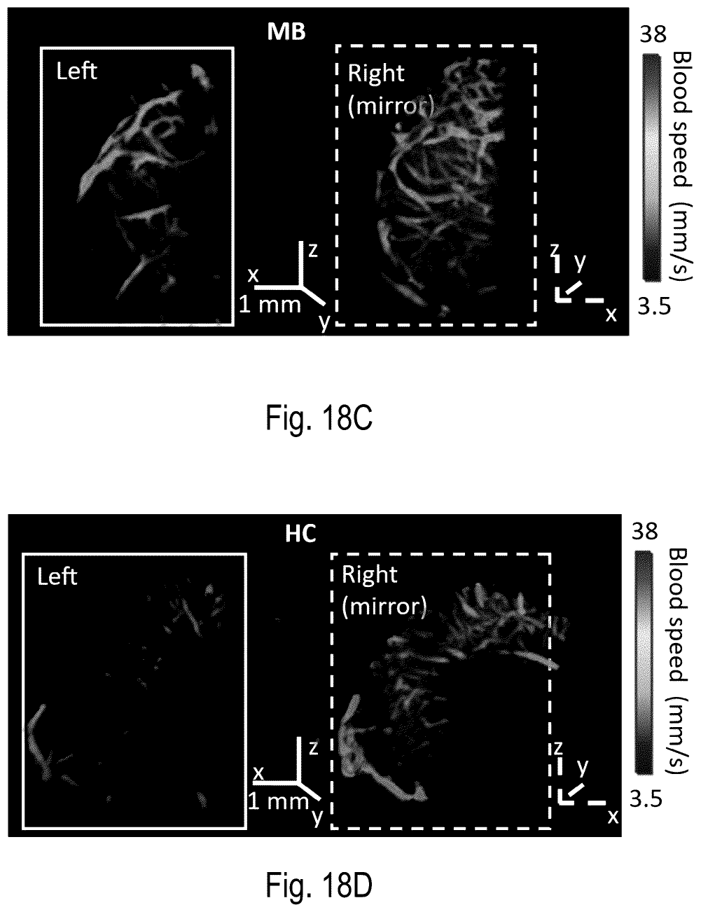

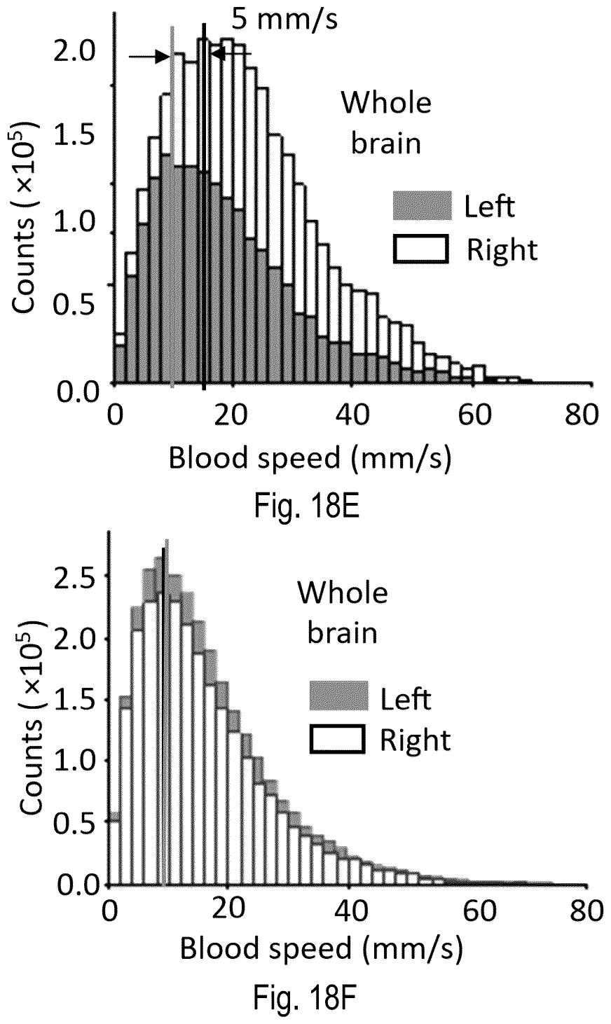

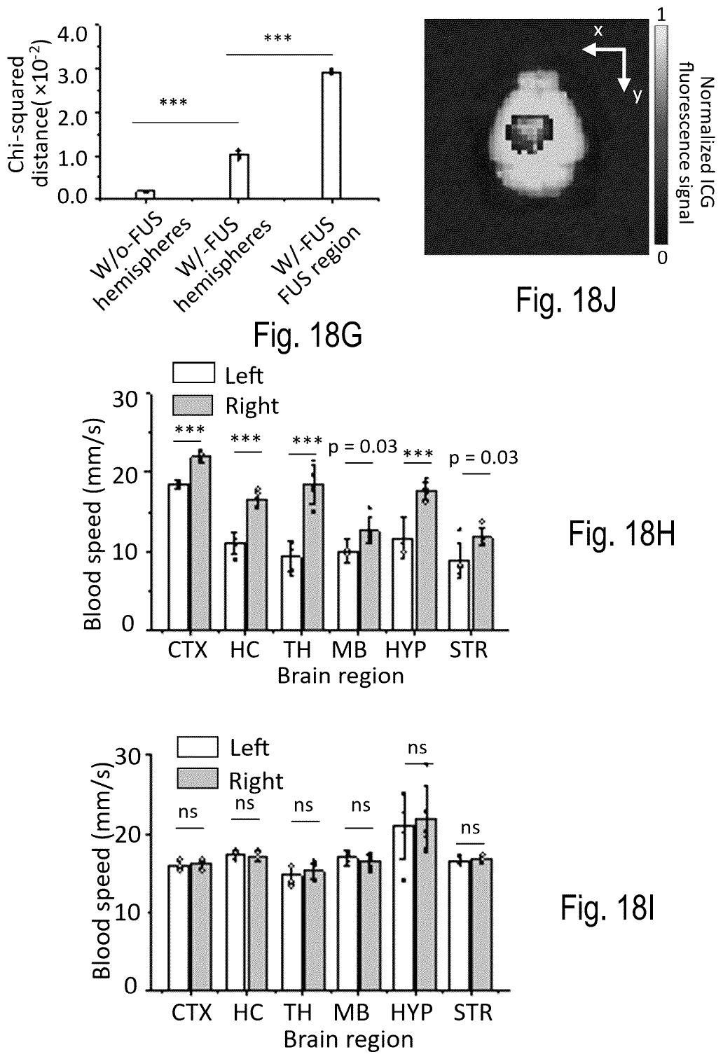

Further, another important hemodynamic parameter, the blood flow speed of the same brain, may be visualized. An overlay of the whole brain blood vascular structure and ICG distribution 110 minutes post-BBB opening (FIG. 18A) with the region from bregma −1.9 to −4.15 mm as a spatial reference (46 slices) may be first plotted. The ICG signal appears in both brain hemispheres due to residual ICG in the blood vessels. However, the left hemisphere, where the BBB was opened, exhibits a higher ICG signal compared to the right hemisphere, as shown in FIG. 14H. The blood flow speed image may then be plotted, and the ultrasound-treated left brain with the untreated right brain may be compared (FIG. 18B). The image shows visible blood flow speed reduction in part of the isocortex, hippocampus, and midbrain of the left brain. In FIG. 18C-18D, the blood flow speed map in single vessel resolution is shown, zooming to the midbrain and hippocampus regions. For a better comparison, the right brain images are flipped. Both areas show visible blood flow speed reduction in the left brain when compared to the corresponding right brain regions.

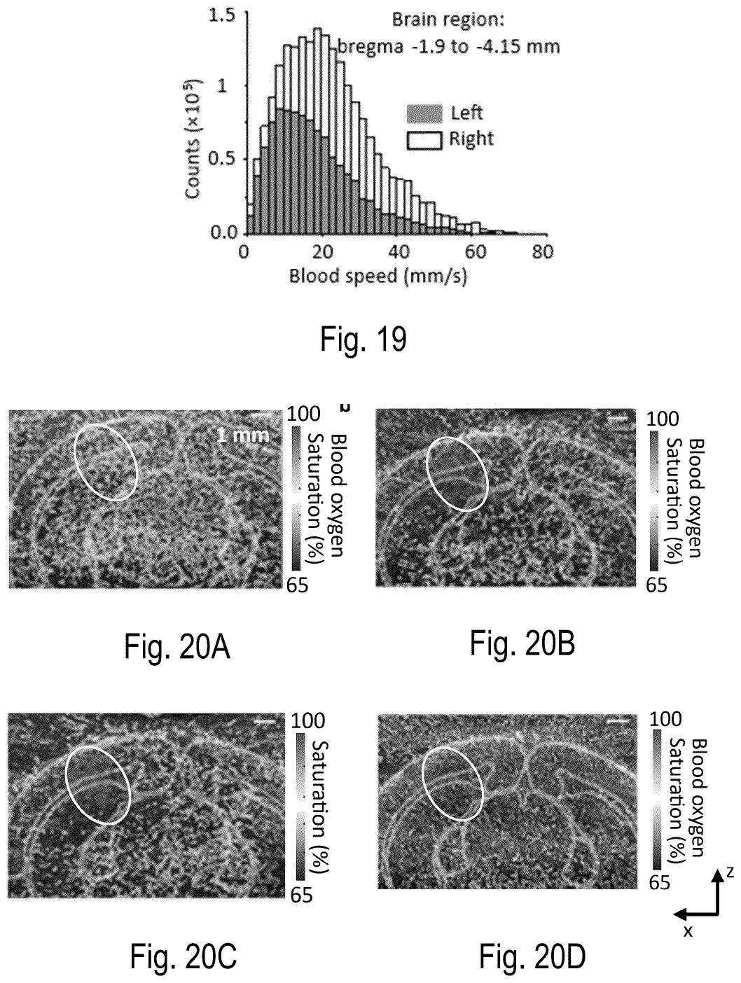

The blood speed distribution in the whole brain hemispheres and the brain region from bregma −1.9 to −4.15 mm at 110 minutes post-BBB disruption is plotted in FIG. 18E, and FIG. 19. In both of the blood speed histograms, lower counts of blood flow speed distribution are observed in the left brain than that in the right brain. Analysis of the blood speed histograms also reveals a ˜5 mm/s left shift of the peak in the left brain in response to BBB disruption. In contrast, the same measurement of cerebral blood speed distribution from healthy mice shows no difference between blood speed distribution in left and right brain regions (FIG. 18F). The similarity of left and right brain blood speed distribution is quantified by calculating the Chi-squared distance of the histograms. As shown in FIG. 18G, the Chi-squared distance between the left and right brain is 0.000176±0.00007 for a brain without FUS treatment, indicating similar blood speed distribution in both hemispheres. The Chi-squared distance is 0.02933±0.00052 for the brain region near FUS exposure and 0.01040±0.00079 between the left and right brain hemispheres, confirming the change in blood speed is caused by FUS-BBB permeability disruption.

The difference in the average blood flow in the specific brain regions is then analyzed. The result in FIG. 18H shows a significant reduction of averaged blood flow in left CTX (15.86±4.47%), HC (33.57±8.61%), TH (48.52±14.22%), MB (20.67±4.77%), HYP (34.11±13.81%) and STR (25.70±11.54%). The large standard deviation from the thalamus could be attributed to the low microbubble counts. The observation that increased BBB permeability induces blood flow reduction aligns with prior studies using optical imaging and MRI. Blood flows through the cerebral vascular network at a rate that is modulated by neurovascular coupling, a set of mechanisms by which the brain recruits additional blood flow to active areas Prior evidence shows that FUS-BBB disruption causes rapid vessel constriction, and the constriction reduces blood flow.

The brain blood oxygenation is also mapped with PAUL imaging before and after FUS treatment (FIG. 20A-20D). The blood oxygenation maps display regions of high and low oxygenation adjacent to each other, which may be influenced by reconstruction noise, particularly in areas with low signal strength. However, these variations occur at the pixel level and do not significantly impact the overall trends in oxygenation changes when integrating the signal across the brain region analyzed in this study. The blood oxygenation maps reveal decreased blood oxygenation in the FUS-exposed area, coinciding with ICG retention. Although it is well documented that low oxygen levels in the blood cause the elevation of the BBB permeability, it is not clear how local acute BBB permeability change affects the blood oxygen, an interesting topic that requires further investigation. To confirm BBB opening, after 4 hours of ICG solution injection, animals are sacrificed and perfused with 1×PBS. Then, brains are excised and imaged using an IVIS spectrum imaging system. The fluorescence image of the harvested brain is shown in FIG. 18J, where a strong ICG fluorescence signal on the left brain confirms the successful BBB disruption with FUS. The higher ICG fluorescence signal in the left-brain region also aligns with ICG PA signal distribution, as shown in FIGS. 14C, E and FIG. 18A.

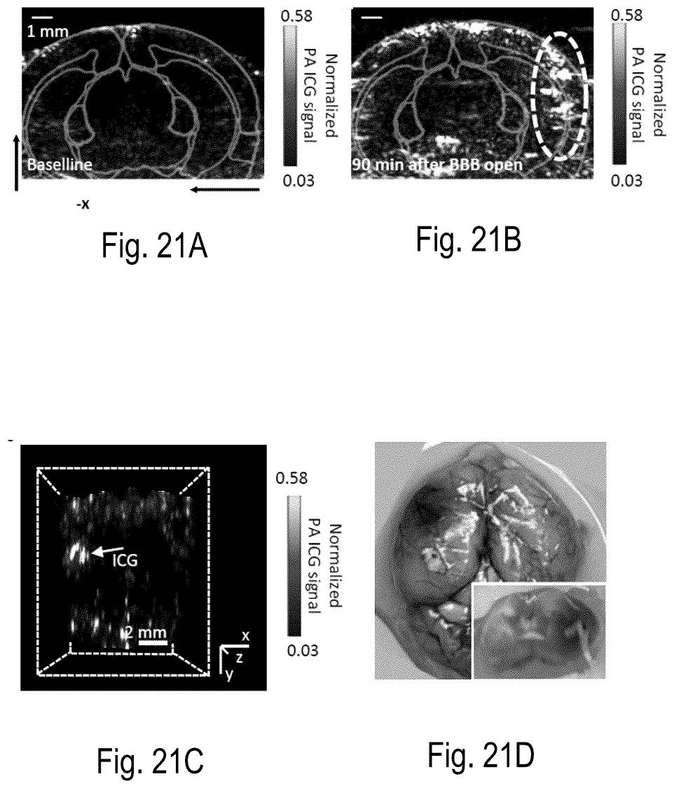

Additionally, to confirm the ICG as a valid tracer, the same BBB-opening experiment but using both ICG and Evans blue (EB) as co-tracers for BBB disruption is repeated. EB, another FDA-approved dye, is the most widely used method to evaluate BBB disruption. EB cannot normally pass through the BBB, and thus, its presence in brain tissue indicates alterations in BBB permeability. The mouse brain with PA imaging is first imaged to record the baseline through the intact skull by repeating the same FUS procedure and then injecting the ICG/EB/PBS through the tail vein. The ICG PA image of the brain before (FIG. 21A) and 90 minutes after the injection (FIG. 21B) may be recorded. The top view of the 3D ICG PA image also clearly reveals highly concentrated ICG accumulated at the left brain region (FIG. 21C). After 4 hours of tracer solution injection, animals are sacrificed. Then, brains are excised and imagined. The photographs (FIG. 21D) show EB dye-stained brain inducing a successful BBB opening. The spatial overlap of the ICG PA signal with the EB-stained confirms that both ICG and EB are effective tracers for evaluating the BBB disruption.

### Imaging System and Imaging Sequence

The 3D PAUL imaging system incorporates a dual-modal design comprising the following key components: a Verasonics ultrasound imaging research system (Vantage 256, Verasonics, Inc., Kirkland, WA, USA), a wavelength tunable (690 nm to 950 nm) OPO laser source with 7-ns-pulse and 10 Hz pulse repletion rate (Phocus Essential, Opotek, Inc., Carlsbad, CA, USA), a customized 15 MHz linear array transducer (Vermon, France) and a XYZ linear stage. The system uses a custom bifurcated fiber bundle to channel light from the laser source. A function generator synchronizes laser pulses with DAQ. To enable side-illumination, the fiber bundle is mounted onto the transducer via a 3D-printed adaptor. During imaging, the transducer is attached to the linear stage and connected to the Verasonics system to capture both ultrasound and PA signals. The acquisition of 3D imaging data is achieved through the mechanical scanning of the transducer across the imaging area.

A hybrid acquisition for the imaging sequence is designed. Each hybrid acquisition includes one PA DAQ and multiple ultrafast ultrasound data acquisition. When the laser system generates a laser pulse, it simultaneously sends a trigger signal to the Verasonics system, and the system starts to receive the generated PA signal. Then, the system transmits and receives multiple ultrasound signals for 100 milliseconds. For each ultrasound frame, the transducer transmits plane waves with seven-angle (−6° to) 6° at a frame rate of 500 Hz. The time interval between two adjacent frames (either PA/US or US/US) is 2 ms. In this case, the acquisition time of dual PAUL imaging is determined by the acquisition time of UL imaging. The speed of PA tomography in the setup is primarily constrained by the laser repetition rate at 10 Hz. It is important to note that this is not a theoretical limit. Higher frame rates up to 7000 Hz have been demonstrated with high repetition rate lasers.

### Characterization of FUS System

In this study, a FUS transducer with a central frequency of 0.5 MHZ (H104, Sonic Concepts, Inc., Bothell, WA, USA) is used. The transducer is driven by the Transducer Power Output system (TPO-105, Sonic Concepts, Inc., Bothell, WA, USA). To create the correct acoustic environment, a cone-shaped water container is designed to hold degassed water, which is assembled with the FUS setup. An acoustic membrane, sealed with acoustic gel, separated the water in the container from the sample. The transducer's focal point is positioned approximately 2 mm from the acoustic membrane.

The transducer is calibrated using a hydrophone scanning system (AIMS III, ONDA Corporation, Sunnyvale, CA, USA) that features a membrane hydrophone (HMB-0500, ONDA Corporation, Sunnyvale, CA, USA). To determine the FWHM of the focal spot, a 2D scan is performed at the focal plane.

### In Vivo PAUL Brain Imaging

The mouse is placed on a heating pad and is anesthetized with 2% isoflurane at 2 L/min of oxygen flow. A 30-gauge catheter is cannulated into the tail vein of a mouse. The animal is then placed in a stereotaxic frame (RWD, China) and the skin above the skull is removed but the skull is kept intact. The acoustic gel is applied on the skull and a home-made plastic water tank with a transparent bottom window is placed above the gel and head. The transducer is placed in the water tank and on the head of the mouse. Microbubbles (Vevo Micromarker) with a concentration of 1×10⁸ bubbles/mL are injected at the rate of 20 μL/min using a syringe pump. A single UL imaging modality is collected using hybrid imaging sequences with PA off for deep learning datasets.

The oxygen challenge of a mouse brain is used in this study with the protocol as follows. Initially, 100% oxygen is used for 100 seconds, followed by a 100-second exposure to a 3% oxygen and 97% nitrogen mixture. This cycle is repeated, and the challenge is concluded with a return to 100% oxygen. During the challenge, the hybrid imaging sequences are continuously to collect dual imaging data with the laser wavelengths 750 nm and 850 nm.

To longitudinal monitor BBB disruption after FUS-mediated BBB opening (see next section for the detailed protocol), the hybrid PAUL imaging sequences are applied before and after 15 min, 30 min, 50 min, 70 min, 90 min, 110 min, and 170 min of ICG solution injection. The transducer is placed in the water tank and on the head of the mouse. Microbubbles with the concentration of 1×10⁸ microbubbles/mL are injected via a catheter at the rate of 20 μL/min only at 110 min of ICG solution injection. For each time point, the transducer is scanned laterally to collect 3D imaging data of the whole brain. For each position, 4 seconds of data acquisition is performed with 4 laser wavelengths of 700 nm, 780 nm, 800 nm, and 850 nm. The step size is 0.05 mm, and a total of 260 positions are recorded.

### FUS-Mediated BBB Disruption

The animals are anesthetized by isoflurane. A 30-gauge catheter is cannulated into the tail vein of a mouse. The animal is then placed in a stereotaxic frame (RWD, China). FUS transducer (central frequency: 0.55 MHZ, H104, Sonic Concept) is positioned on top of the mouse head, and the foci of the ultrasound beam is located to the left mouse brain. The cone-shaped water container is assembled using the FUS setup and filled with degassed water. An acoustic membrane is employed to seal the interface between the water and the mouse brain, and it is coupled with acoustic gel. About 50 μL of microbubble solutions (2×109 bubbles/mL) are injected via the mouse tail vein. After 1 min of microbubble injection, FUS is turned on to induce BBB disruption. The following FUS parameters are applied for the BBB disruption, 0.2 MPa negative peak pressure, 2 ms of burst length, 1 Hz of pulse-repetition rate, 60-seconds of sonification on and 60-second sonification off; this cycle repeated once. After FUS treatment, 100 μL ICG (Adooq Bioscience, USA) solution with a concentration of 7 mg/mL is injected via the mouse tail vein to monitor BBB disruption.

To confirm BBB opening, after 4 hours of ICG solution injection, animals are sacrificed and perfused with PBS. Then brain tissue is excised and imaged using an in vivo epic-fluorescence system (IVIS spectrum imaging system). Specifically, imaging is recorded with an excitation filter centered at 745 nm, an emission filter centered at 840 nm, and 2 seconds of exposure time. Additionally, for tracking BBB opening using ICG/EB mixed tracers, 150 μL of mixed ICG and EB solution (0.7 mg ICG and 2 mg EB in PBS) is injected via the mouse tail vein. After PAUL imaging, the mouse is sacrificed, and the mouse brain is excised at the end of the imaging to photograph the EB-staining to confirm its extravasation.

### Histological Processing

FUS with different conditions was applied to induce BBB disruption. Following the FUS treatment, 100 μL of Evans Blue solution (18 mg/mL) was injected via the tail vein to visualize the BBB permeability change. After two hours of FUS-medicated BBB disruption, the mice were sacrificed, and transcardiac perfusion was performed with 60 mL of phosphate-buffered saline (PBS) for 24 minutes, followed by 60 mL of 10% neutral buffered formalin (NBF) for another 24 minutes. The brains were then extracted and stored in NBF solution for two days in preparation for paraffin sectioning. Brain regions containing Evans Blue signal were embedded in paraffin and sectioned into 5 μm thick slices along the coronal plane. The brain slices are strained with hematoxylin and eosin (H&E) and TUNEL (with Click-iT™ TUNEL Alexa Fluor™ assay) staining to look for the signs of hemorrhage and neuronal apoptosis, respectively. The H&E slices were scanned by PhenoCycler-Fusion (CODEX) System with 4× magnification. The TUNEL stained slices were scanned by BioTek Cytation 5 Multi-Mode Reader with 4× magnification.

### Post-Imaging Processing for Power Doppler Images

The acquired ultrasound data are beamformed using the delay and sum algorithm to reconstruct ultrasound images. By applying the SVD based spatiotemporal filtering to reconstruct the ultrasound cineloop, a Doppler signal is obtained over time. Then, a rigid motion model is used to correct the tissue's motion. Briefly, a frame-to-frame rigid transformation is applied to ultrasound image frames, and the translation and rotation information is calculated using gradient descent optimization (imregtform function with regular step gradient descent optimizer in MATLAB). Doppler signal is corrected to compensate for tissue motion using translation and rotation information. The power Doppler image is calculated as the summation of the Doppler intensity.

### Post-Imaging Processing for UL Images

First, ultrasound data are beamformed using the delay-and-sum algorithm to create reconstructed images. Specifically, images with different tilted plane wave transmissions were spatially and coherently summed to produce a fully dynamically focused image. While visualizing individual microbubbles does not require multiple-plane wave compounding, compounding significantly enhances imaging contrast and SNR. Without compounding, the localization accuracy in UL imaging can be greatly reduced. It is important to balance the number of angles used for compounding. Using too many angles can reduce the frame rate of ultrafast ultrasound imaging, potentially affecting the detection of dynamic blood flow. In practice, 3-9 angles are typically used to maintain a balance between frame rate and image quality, and in this study, we selected 7 angles

To extract signals from flowing microbubbles, spatiotemporal filtering based on SVD is applied. Microbubbles, significantly smaller than the ultrasound wavelength, can be distinctly identified in space and time, appearing as Point Spread Functions (PSFs) in the ultrasound system. To estimate the system's PSF, Gaussian fitting on signals from isolated microbubbles is performed. Following this, cross-correlation between the PSF and signals from microbubbles across consecutive frames is calculated to identify microbubble locations. A cross-correlation coefficient greater than 0.7 indicates isolated blobs, with the peak in each blob pinpointing the location of microbubbles in each frame. Tissue motion is corrected using a motion compensation model, which is consistent with those employed in power Doppler processing. The final UL image is generated by accumulating the identified microbubble positions over multiple frames.

To determine blood velocity, the movement of microbubbles is tracked using the Hungarian tracking algorithm. This algorithm computes distances between every pair of microbubbles in the current frame and the next, minimizing the total distance to establish optimal connections between microbubbles in successive frames. By applying this algorithm across all frames, a set of microbubble tracks is created over time. The velocity of each microbubble is calculated by differentiating its position in the trajectory relative to the time vector.

### Deep Learning Model

The DL network is adapted from pix2pix architecture, a special cGAN for image-to-image translation, and A-net, which is a modified pix2pix network used for super-resolution optical microscopy. It includes two main networks: a generator and a discriminator. The generator relies on U-net architecture to reconstruct super-resolved ultrasound images. The U-net takes 512×512 sized sparse UL images and power Doppler images as inputs. The encoder is built on convolution layers with a stride of 2 for down-sampling, followed by batch normalization and leaky ReLUs for activation. Skip layer connections are added to the encoder networks to concatenate feature maps located symmetrically between the encoder and the decoder. The decoder network upsamples every input using convolutional layers, resulting in feature maps of a double on each dimension and half in the number of channels. The input is upsampled by a factor of 2 using nearest-neighbor interpolation followed by a convolutional layer. The last layer of the decoder network uses a Tanh activation function to output the reconstructed dense UL image with the size of 512×512. Batch normalization is used after all convolutional layers with dropout units with a probability of p=0.5 applied to the second, third, and fourth layers of the decoder. Skip-layer connections are added to the generator to symmetrically link convolution and deconvolution layers with the same size, passing the local and pixel-wise context from the encoder network to the decoder network.

The discriminator network comprises five convolutional layers and reduces the output to a size of 64×64. The discriminator takes a 3-channel image as an input: the sparse UL image, the power Doppler image, and either the reconstructed or real dense UL image. The pixel values of the down-sampled output image indicate whether the third input image channel is a real dense UL image, or an image produced by a generator.

For the input data preparation, the Power Doppler images, sparse UL images, and complete UL images were acquired from different mice to introduce variability. We first recorded the “complete data set” by a long DAQ time and used the whole dataset to produce a ground truth UL image with complete (saturated) vessel structures, where the images were reconstructed by the traditional low-speed UL imaging reconstruction method. We then randomly selected the partial length ( 1/20- 1/25) of the data from the same dataset to produce a low-resolution power Doppler image of the vessels and the corresponding UL image. Because only part of the data was used to produce the input UL image, its vessel structures were not completely saturated and looked sparse. Since the input power Doppler and sparse UL image, as well as the ground truth UL image, were produced from the same imaging recording, they were paired and used as the inputs for training the network.

The dataset was then divided into 10,240 paired image patches of size 572×572 pixels, which were further randomly cropped to 512×512 during training for data augmentation. Since Power Doppler and sparse UL signals are influenced by blood flow, variations in blood flow rate are accounted for during training. The inclusion of data from different mice naturally introduced slight variations in blood flow. While it is challenging to collect sufficient training data from mice with significantly altered blood flow rates (e.g., due to BBB disruption or oxygen challenge tests), this is addressed by randomly selecting microbubble events across the acquisition time to generate sparse UL and power Doppler images. This approach simulates different blood flow rates in the training dataset.

The objective of cGAN can be expressed as:

$${L}_{cGAN} ( G , D ) = arg {min}_{G} {max}_{D} ( {𝔼}_{x , y} [ log D ( x , y ) ] + {𝔼}_{x , z} [ log ( 1 - D ( x , G ( x , z ) ) ) ] ) .$$

In the study above, the input x is the sparse UL image S combined with the up-sampled power Doppler image P. The target y is the dense UL image T. Similar to pix2pix implementation, the noise z is only applied through dropout layers. Therefore, the objective function for the cGAN is rewritten as,

$${L}_{cGAN} ( G , D ) = arg {min}_{G} {max}_{D} ( {𝔼}_{S , P , T} [ log D ( S , P , T ) ] + {𝔼}_{S , P , z} [ log ( 1 - D ( S , P , G ( S , P ) ) ) ] ) ,$$

To improve the quality of the reconstructed image, an additional objective for the training of the generator G is incorporated. The combination of multiscale structural similarity index (MS-SSIM) and L1 norm are applied to minimize the difference between generator output \hat{T} and the real dense UL image T, expressed as,

$${L}_{S R} ( G ) = {𝔼}_{( S , P , T )} [ ρ ( 1 - MSSSIM ( G ( S , P ) , T ) ) + ( 1 - ρ ) \vert \vert G ( S , P ) - T {\vert \vert}_{1} ] ,$$

The scaler weight ρ is set to 0.84, as in ref. In summary, the final objective function can be defined as the following,

$${G}^{⋆} = arg {min}_{G} {max}_{D} α {L}_{S R} ( G ) + γ {L}_{c G A N} ( G , D ) .$$

The weights are manually set: α=50 and γ=1. The objective function is optimized with stochastic gradient descent (SGD) and Adam optimizer at the batch size of 30. The network is trained for 20,000 iterations using a single NVIDIA 2080 Ti GPU.

### Post-Imaging Processing for DL-UL Imaging

DL-UL images are generated by feeding sparse UL images and power Doppler images into the trained cGAN (described in the deep learning model section). Here, a sparse UL image means the image is generated using a short acquisition time (4 sec of data acquisition). A sparse UL image is generated following the UL imaging workflow. power Doppler image is generated with the same datasets of sparse UL image following power Doppler imaging workflow.

### Post-Imaging Processing for PA Imaging

After PA DAQ, reconstructed PA images are constructed using the delay and sum algorithm. We used MCXLab simulator to model the light propagation. Different light propagation layers (i.e. air, skin, tissue, and skull) from ultrasound volumes are manually segmented using a 3D slicer. Then, the bi-side light illumination is simulated using two planer beams with an angle of 30 degrees. The optical properties of different components are assigned similarly. The fluence map obtained from the Monte Carlo simulation is then used to compensate for the PA images.

Linear regression-based spectrum unmixing is performed to extract each contrast signal from compensated PA images. Briefly, the measured PA signal is a linear combination of the spectral signatures of different chromophores (i.e. hemoglobin, deoxyhemoglobin and ICG) with the relative concentration of each chromophore.

In the implementation, the absorption spectrum is first obtained either by measurement or database (i.e. the absorption of hemoglobin and deoxyhemoglobin is from https://omlc.org/spectra/hemoglobin/). The following linear equation is used to estimate the molar concentration of different contrast components:

$$[ \begin{matrix}{C}_{HbR} ( x , y ) \\ {C}_{{HbO}_{2}} ( x , y ) \\ {C}_{ICG} ( x , y )\end{matrix} ] = {( {ε}^{T} ε )}^{- 1} {ε}^{T} {P}^{′} ,$$

where P′ is the PA signal after fluence compensation.

The oxygen saturation is calculated by:

$${s O}_{2} ( x , y ) = \frac{{C}_{H b {O}_{2}} ( x , y )}{{C}_{H b {O}_{2}} ( x , y ) + {C}_{H b R} ( x , y )} .$$

### Brain Allen Atlas Registration

The registration of the PAUL image with the Allen Mouse Common CoordinatFramework s performed with the registration software described in. Briefly, a power Doppler volume is first generated from PAUL data. Manually translating, scaling, and rotating the power Doppler volume is required to align Allen Mouse Common Coordinate Framework. The software outputted an affine transformation matrix after manual registration. Then, this transformation could be applied to UL or PA volumetric map and used to extract signals from any regions of interest for quantification.

### Data and Statistical Analysis

MATLAB is used to process signals and images. For the image display, the ultrasound images are shown in logistic scale, PA, doppler, and UL images are displayed in linear scale. The pixel size of ultrasound and PA images is 50 μm. The pixel size of UL or DL-UL image is 10 μm. 3D volume with distance color encoded is rendered with 3D PHOVIS, and the rest of the 3D volume is rendered with Amira 2022.1. Data is plotted using Origin 2020.

The resolution of UL images is measured either by amplitude profile of vessels or Fourier Ring Correlation (FIG. 3 and FIG. 5A-5B). To measure the resolution of 2D UL and DL-UL images, 9 vessels are selected, and the cross-section of each vessel is measured. The resolution is calculated as the FWHM of the cross-section curve. To measure the resolution of 2D sparse UL images, 9 regions with the size of 512×512 are randomly selected. The list of tracking in these regions is randomly separated into two parts, and two super-resolution images are generated based on two tracking data. Then, the normalized correlation of two spectra of these images is calculated, and a fixed threshold (½ bit) is used to determine the resolution. In addition, to evaluate the difference in resolution of complete UL, sparse UL, and DL-UL, the two-tailed p-value is calculated using an unpaired student t-test to determine the significance. The data are considered to be statistically significant with p<0.05.

To segment brain images into different regions, binary masks corresponding are computed to six different regions of the brain (Isocortex, Hippocampus, Midbrain, Striatum, Thalamus, and Hypothalamus) based on Allen Mouse CCF. The masks are then applied to either microbubble density/counts map, vascular map, or PA image, allowing them to split and represent different biological information for each region.

To measure the blood speed profile, the flow velocity profile is analyzed across the sections of different vessels (FIG. 8). 10 sections of each vessel are selected, and the velocity profile of a vessel is plotted as the distance versus the velocity speed. Spatial variations of velocity profiles are analyzed using an unpaired student's t-test to evaluate the significance of the difference.

To quantitatively evaluate BBB disruption, ICG signal over time at the left and right hemispheres is generated. Standard deviation is calculated by separating the regions into 3 sections and calculating ICG signal in each section. The brain volume is then cropped into the FUS sonification region (bregma −1.9 mm to −4.15 mm) and brain binary masks are applied to separate the region into 6 brain sub-regions (Isocortex, Hippocampus, Midbrain, Striatum, Thalamus, and Hypothalamus). The blood velocity, ICG concentration and microbubble counts in these subregions are calculated. To evaluate the signal difference in different subregions, the two-tailed p-value is calculated using an unpaired student t-test to determine the significance.

In the various implementations above, a multi-parametric DL-enabled PAUL imaging is described and is utilized to concurrently assess dynamic responses of oxygenation, brain hemodynamic changes, and BBB permeability throughout the entire mouse brain with high resolution and deep penetration depth. The DL approach acceleration improves imaging speed 25-fold over conventional UL imaging without the need for high-concentrated microbubble injections. The increase in speed without increasing microbubble concentration is crucial for 3D scanning of the entire brain using a clinically relevant linear array transducer, as it extends the maximum allowable imaging duration and minimizes imaging artifacts, which are extremely challenging with traditional UL or other DL-acceleration UL methods.

Using this technology, the first in vivo visualization of the entire mouse brain microvasculature with a linear array transducer may be achieved, resolving microvascular structures as small as 22 μm. This example approach achieves acquisition speeds and spatial resolution comparable to 3D UL imaging performed with matrix arrays, while significantly reducing system complexity and cost. Additionally, detailed blood flow dynamics may be tracked within individual microvessel and quantified changes in blood velocity and flow rate across a living mouse's brain at the single microvasculature level in response to varying levels of oxygen inhalation are captured and quantified. The implementations above establish a direct relationship between oxygen delivery and subsequent vascular reactivity, highlighting brain regions that undergo significant changes in key hemodynamic parameters during oxygen challenge tasks.

Further, by harnessing the multi-parametric advantages of DL-enhanced PAUL imaging, its effectiveness as a new imaging tool to guide and evaluate FUS-mediated BBB opening is demonstrated. It is shown that DL-enhanced PAUL imaging can non-invasively map the diffusion of tracer molecules crossing the BBB and observe the subsequent cerebral vascular responses following BBB permeability modulation in one imaging acquisition. The observed decrease in blood flow and flow rate, particularly in regions with elevated tracer retention, indicates hemodynamic responses to BBB opening. It is also found that BBB modulation causes a relatively global impact on cerebral hemodynamics beyond FUS exposure regions, despite each region showing different flow responsibility to the disruption. These changes support the hypothesis that FUS-induced BBB disruption leads to a cascade of vascular responses, potentially having immediate and lasting effects on CNS function. Quantitative analyses of the high-resolution imaging data confirm PAUL imaging's ability to segment and analyze microvascular structures and functions in key anatomical regions. It provides a framework for understanding the diverse responses of these areas to BBB disruption.

The imaging technique above is designed primarily for preclinical FUS-induced BBB studies with clinical translation in mind. A linear array transducer is chosen due to its compatibility with ultrafast plane-wave imaging essential for super-resolution ultrasound imaging and its cost-effectiveness and adaptability to clinical systems. There may still be challenges for clinical translation, including skull-induced acoustic aberrations, limited penetration depth in PA imaging, and spectral coloring effects. Several example further solutions may be developed. For instance, techniques such as full-wave inversion and adaptive focusing can correct human skull-induced aberrations. Additionally, emerging biomaterials with acoustic properties that match soft tissue can be used to replace parts of the skull, effectively reducing aberration effects. Improvement in wavefront shaping, high-sensitivity transducers, and strong PA tracers can be employed to enhanced PA imaging penetration. Furthermore, Monte Carlo simulations and machine learning approaches may be used to mitigate spectral coloring. These challenges represent technical, rather than fundamental, barriers. Development in transducer technology, image reconstruction, and light delivery may support the clinical adaptation of this dual-modal imaging approach for safe and effective FUS BBB opening.

An example advantage of the PAUL system is its operational flexibility. In some example implementations, imaging can be performed in dual PAUL mode, PA-only, or UL-only mode, accommodating diverse experimental and clinical needs. While the current example demonstration shows single-time microbubble injections in PAUL mode due to dose limitations-common to all UL imaging, the system above allows for versatile protocols. For instance, small doses of microbubbles could be used for quick 2D imaging at any time to monitor BBB status in terms of hemodynamic and oxygenation. For more detailed analyses, dual-modal 3D imaging can be performed at specific time points. Additionally, when the FUS device is co-aligned with the imaging probe, the example system above has the potential to monitor hemodynamic changes in real-time during BBB disruption using microbubbles for FUS activation. High-concentration microbubbles could potentially cause shadowing effects when combined with FUS for simultaneous therapy and imaging. This challenge further underscores the advantage of the DL-enhanced PAUL imaging above, which achieves effective imaging with only low concentrations of microbubbles. Although cavitation of microbubbles during BBB opening could influence hemodynamic measurements, this effect is minor and does not significantly impact the performance of the dual imaging methodology above. The measurements are only affected within the FUS focal region where microbubble destruction occurs. Hemodynamic changes outside the focal area can still be reliably monitored by tracking microbubbles. Additionally, by redesigning the imaging sequence with short, cyclic bursts of FUS activation, this adjustment enables us to monitor blood flow information even in the focal region during the intervals when FUS is off. A time point of BBB disruption monitoring may be demonstrated to showcase the effect, and this approach is flexible and can enable high-resolution imaging and comprehensive analysis at critical stages.

The example implementations above present a significant advancement in neuroimaging through the development and application of DL-accelerated PAUL imaging. It is demonstrated that this multi-parametric dual-modal imaging approach allowed for the simultaneous visualization of the BBB's permeability changes and detailed cerebrovascular mapping, capturing the complexities of the brain's vascular response to FUS treatment.

It is to be understood that the various implementations above are not limited in its application to the details of construction and the arrangement of components set forth above and in the accompanying drawings. The disclosure is intended to cover other embodiments that may be practiced or carried out in various ways following the underlying principles disclosed herein.

It should also be noted that a plurality of hardware and software-based devices, as well as a plurality of different structural components, may be used to implement the various embodiments of the disclosure. In addition, it should be understood that embodiments of this disclosure may include hardware, software, and electronic components or modules that, for purposes of discussion, may be illustrated and described as if the majority of the components are implemented solely in hardware. However, one of the ordinary skills in the art, and based on a reading of this disclosure, would recognize that, in at least one embodiment, the electronic-based aspects of the invention may be implemented in software (e.g., stored on a non-transitory computer-readable medium) executable by one or more processors. As such, it should be noted that a plurality of hardware and software-based devices, as well as a plurality of different structural components, may be utilized to implement the invention. Furthermore, and as described in subsequent paragraphs, the specific mechanical configurations illustrated in the drawings are intended to exemplify embodiments of the invention and that other alternative mechanical configurations are possible. For example, “controllers” described in the specification can include standard processing components, such as one or more processors, one or more computer-readable medium modules, one or more input/output interfaces, and various connections (e.g., a system bus) connecting the components. These controllers may be implemented as dedicated processing circuitry or in general-purpose processors, in combination of various software and/or firmware, and in combination of other wired or wireless communication interfaces.

We claim:

1. A method for imaging a target region surrounding a microvessel for blood, comprising:
generating microbubbles in the microvessel having a low concentration in the target region;
introducing a plurality of tracer agents in the microvessel in the target region;
generating a sequence of ultrasound images based on ultrasound response from the microbubbles, the sequence of ultrasound images indicating a blood flow evolution in the microvessel;
generating a sequence of optical pulses to induce acoustic responses from at least the plurality of tracer agents and
detecting the acoustic responses to form a sequence of photoacoustic images to monitor a diffusion evolution of the tracer agents through the microvessel, the sequence of photoacoustic images being time-interleaved with the sequence of ultrasound images at a frame rate of at least 10 frames per second.

2. The method of claim 1, wherein the acoustic responses are detected by an array of acoustic detectors to generate the sequence of photoacoustic images.

3. The method of claim 2, wherein the array of acoustic detectors are activated by a triggering signal synchronized with the sequence of optical pulses.

4. The method of claim 1, wherein the sequence of optical pulses comprises at least a first spectral component aligned with an absorption line or band of the plurality of tracer agents to generate imaging information for the diffusion evolution of the tracer agents.

5. The method of claim 4, wherein:
the sequence of optical pulses further comprises a second set of spectral components; and
the sequence of photoacoustic images further comprise oxygenation evolution information generated by the second set of spectral components.

6. The method of claim 5, wherein the oxygenation evolution information is generated differentially from photoacoustic response from oxygenated and deoxygenated states of hemoglobin.

7. The method of claim 1, wherein the sequence of ultrasound images of the microbubbles are generated from the ultrasound response of the microbubbles based on an ultrasound localization technique.

8. The method of claim 7, wherein generating the sequence of ultrasound images comprises:
generating a sequence of original ultrasound images from the ultrasound response of the microbubbles using the ultrasound localization technique; and
processing the sequence of original ultrasound images using a pre-trained deep learning model to generate the sequence of ultrasound images with enhanced spatial resolution over the sequence of original sequence of ultrasound images.

9. The method of claim 8, wherein the pre-trained deep learning model comprises neural network.

10. The method of claim 9, wherein the neural network comprises a U-Net comprising a down-sampling encoder network followed by an up-sampling decoder network.

11. The method of claim 1, wherein:
the target region comprises a cerebrovascular region; and
the microvessel comprises a blood-brain-barrier (BBB).

12. The method of claim 11, further comprising applying a focused ultrasound signal to a portion of the BBB prior to generating the sequence of blood-flow images and the sequence of photoacoustic images.

13. The method of claim 12, wherein:
the focused ultrasound signal generates an opening in the BBB; and
the sequence of photoacoustic images indicates the diffusion evolution of the tracer agents through the opening in the BBB.

14. The method of claim 1, wherein the target region is non-superficial.

15. The method of claim 14, wherein the target region is at least 1 centimeter deep under skin.

16. The method of claim 1, wherein the microbubbles are generated at a concentration of less than 10⁷ per ml.

17. A system for imaging a target region surrounding a microvessel for blood, comprising:
an injection subsystem configured to generate microbubbles in the microvessel having a low concentration in the target region, and to introduce a plurality of tracer agents in the microvessel in the target region;
an ultrasound imaging subsystem configured to generate a sequence of ultrasound images based on ultrasound response from the microbubbles, the sequence of ultrasound images indicating a blood flow evolution in the microvessel;
an optical subsystem configured to generate a sequence of optical pulses to induce acoustic responses from at least the plurality of tracer agents; and
an array of acoustic detectors configured to detect the acoustic responses to form a sequence of photoacoustic images to monitor a diffusion evolution of the tracer agents through the microvessel, the sequence of photoacoustic images being time-interleaved with the sequence of ultrasound images at a frame rate of at least 10 frame per second.

18. The system of claim 17, wherein:
the sequence of optical pulses comprises at least a first spectral component aligned with an absorption line or band of the plurality of tracer agents to generate imaging information for the diffusion evolution of the tracer agents;
the sequence of optical pulses further comprises a second set of spectral components; and
the sequence of photoacoustic images further comprises oxygenation evolution information generated by the second set of spectral components.

19. The system of claim 17, wherein the ultrasound imaging subsystem is configured to generate the sequence of ultrasound images by:
generating a sequence of original ultrasound images from the ultrasound response of the microbubbles using an ultrasound localization technique; and
processing the sequence of original ultrasound images using a pre-trained deep learning model to generate the sequence of ultrasound images with enhanced spatial resolution over the sequence of the original sequence of ultrasound images.

20. The system of claim 17, wherein:
the target region comprises a cerebrovascular region;
the microvessel comprises a blood-brain-barrier (BBB); and
the system further comprises a focused ultrasound excitation system configured to apply a focused ultrasound signal to a portion of the BBB prior to generating the sequence of blood-flow images and the sequence of photoacoustic images.
